(19) United States
(12) Patent Application Publication
(10) Pub. No.: US 2023/0398091 A9
(43) Pub. Date: Dec. 14, 2023

(54) COMPOUNDS AND METHODS FOR FORMING ION CHANNELS IN BIOLOGICAL MEMBRANES

(71) Applicants: Washington University, St. Louis, MO (US); Wisconsin Alumni Research Foundation, Madison, WI (US)

(72) Inventors: Baron Chanda, St. Louis, MO (US); Willy Carrasquel Ursulaez, St. Louis, MO (US); Jennifer Schomaker, Madison, WI (US); Corey Jones, Linden, TN (US); Mahzad Dehghany, Madison, WI (US)

(73) Assignees: Washington University, St. Louis, MO (US); Wisconsin Alumni Research Foundation, Madison, WI (US)

(21) Appl. No.: 17/881,038

(22) Filed: Aug. 4, 2022

Related U.S. Application Data
(60) Provisional application No. 63/229,265, filed on Aug. 4, 2021.
(60) Provisional application No. 63/275,987, filed on Nov. 5, 2021.

(51) Int. Cl. A61K 31/357

(52) U.S. Cl. CPC A61K 31/357

(57) ABSTRACT

Self-assembling compounds for the formation of ion channels in biological membranes include monoacylated benzo(crown-ether) (MAcBCE) compounds and monoalkylated benzo(crown-ether) (MAkBCE) compounds. Methods of preparing the MAcBCE and MAkBCE compounds and methods of forming an ion channel in a biological membrane are also disclosed.

### CROSS-REFERENCE TO RELATED APPLICATIONS

This application claims priority to U.S. Provisional Patent Application No. 63/275,987, filed Nov. 5, 2021, and U.S. Provisional Patent Application No. 63/229,265, filed Aug. 4, 2021, the content of each of which is hereby incorporated herein by reference in its entirety.

### STATEMENT REGARDING FEDERALLY SPONSORED RESEARCH OR DEVELOPMENT

This invention was made with government support under GM131662, NS081293, and NS116850 awarded by the National Institutes of Health. The government has certain rights in the invention.

### FIELD OF THE INVENTION

The present disclosure generally relates to self-assembling compounds for the formation of ion channels in biological membranes, methods of preparing the compounds, and methods of forming ion channels in biological membranes.

### BACKGROUND OF THE INVENTION

Crown ethers are cyclic molecules with high affinity to cations. Since Charles Pederson's seminal discovery of organic crown ethers as complexing agents for the binding of alkali metal cations, these compounds have had a tremendous impact in disciplines as far-ranging as organic synthesis, electrochemistry, phase-transfer catalysis, and microbiology. Crown ethers have been used for ion transport through bulk liquid membranes, as sensors and scaffolds for materials, and in the design of biologically active ionophores. The initial motivation in biology for developing synthetic ionophores was to create a new class of antibiotics inspired by many naturally occurring ionophoric antibiotics. Unfortunately, many of the potent ionophores developed in this context also proved highly toxic to the host cells. More recently, research focused on new classes of synthetic ion channels has been spurred by a multitude of factors. First, the exquisite selectivity and ion transport properties of natural ion channels have challenged synthetic chemists to mimic these properties by designing new classes of small molecule compounds. Second, the total synthesis of these compounds enables limitless customizations to precisely test the mechanism of ion selectivity and permeation. Finally, for biologists, synthetic ion channels have potential as useful reagents for physiological studies and as therapeutics for excitability disorders, especially if their activity can be regulated both spatially and temporally.

At present, the architectures of synthetic ion channels can be broadly classified as either unimolecular or supramolecular assemblies. Unimolecular channels include cyclodextrins, pillarene, and helical oligomers, whereas the ion channel activities exhibited by benzo(crown-ethers), cyclic peptides and aromatic macrocycles are due to the formation of supramolecular aggregates. Unimolecular compounds tend to have a more well-defined functional behavior but usually require complex multi-step syntheses. In contrast, supramolecular aggregates offer simpler synthesis, modular designs that enable tunability and are more amenable to controlled activation. Nevertheless, balancing the aggregation properties and membrane partitioning of these compounds can be especially challenging, as the hydrophobicity that is essential for membrane partitioning limits their dissolution in aqueous solutions.

To date, several examples of unimolecular and supramolecular assemblies of synthetic ionophores incorporating crown ethers have been reported, these compounds are envisioned to form stacked arrays, where the macrocycles are oriented parallel to the membrane to allow sequential passage of ions. Two well-characterized supramolecular crown ethers are hexyl-benzoureido-15-crown-5-ether and hexyl-benzoureido-18-crown-6-ether, both of which utilize H-bonding networks to form columnar self-assembled ion channels within lipid bilayers. However, the functional characterization of these compounds is mostly based on flux assays, which typically assays the response of a fluorescent dye to ion transport across the membrane and are therefore an indirect measure. This assay is limited because it does not discriminate between membrane-lytic crown ethers and true ion channels. A more sophisticated, bacteria-based assay has found that many prior characterized benzo(crown-ethers) will cause membrane lysis and are highly cytotoxic. For instance, one family of crown ethers, dialkylated lariat ethers which were previously assumed to form membrane channels based on flux assays, were found to be membrane-lytic and did not exhibit channel activity in lipid bilayers, as reported in Carrasquel-Ursulaez, W., et al., Re-evaluation of the mechanism of cytotoxicity of dialkylated lariat ether compounds, RSC Advances, 10(66), pp. 40391-40394 (2020), and Supplementary Information, pp. S1-S37, the disclosure of which is hereby incorporated herein by reference in its entirety.

Despite these advances in the synthesis of small-molecule biologically active ionophores, the current understanding of their mechanism of action remains limited due to a lack of rigorous functional characterization and direct methods to probe the structures in lipidic environments.

### SUMMARY OF THE INVENTION

Among the various aspects of the present disclosure is the provision of a self-assembling compound for the formation of ion channels in biological membranes. The self-assembling compound is one of a monoacylated benzo(crown-ether) (MAcBCE) compound and a monoalkylated benzo(crown-ether) (MAkBCE) compound.

In some aspects, the MAcBCE compound has the formula (IA):

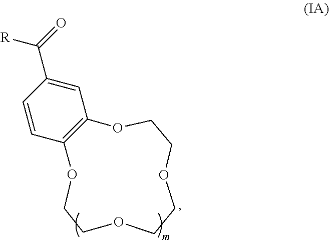

R being a straight chain or branched C₁₋₂₀alkyl, optionally containing unsaturation, that is not substituted with a hydrogen bond donor, and

m being an integer from 1 to 3.

In some aspects, the MAcBCE compound has a formula (IB):

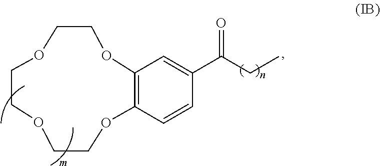

m being an integer from 1 to 3, and

n being an integer from 0 to 19.

In some aspects, the MAcBCE compound has the formula (IB), m is an integer from 1 to 3, and n is an integer from 2 to 9.

In some aspects, the MAcBCE compound has the formula (IB), m is an integer from 1 to 3, and n is an integer selected from the group consisting of 2, 4, 6, 8, and 9.

In some aspects, MAkBCE compound has a formula (IIA):

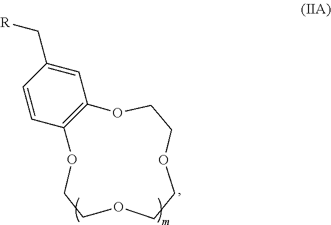

R being a straight chain or branched C₁₋₂₀alkyl, optionally containing unsaturation, that is not substituted with a hydrogen bond donor, and

m being an integer from 1 to 3.

the MAkBCE compound has a formula (IIB):

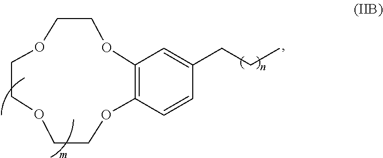

m being an integer from 1 to 3, and

n being an integer from 0 to 19.

In some aspects, the MAkBCE compound has the formula (IIB), m is an integer from 1 to 3, and n is an integer from 2 to 9.

In some aspects, the MAkBCE compound has the formula (IIB), m is an integer from 1 to 3, and n is an integer selected from the group consisting of 2, 4, 6, 8, and 9.

In various aspects of the present disclosure, methods of preparing benzo(crown-ether) compounds being monosubstituted with one of an acyl group and an alkyl group are provided. The methods include reacting a carboxylic acid having a formula (III):

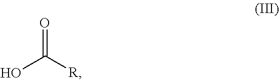

R being a straight chain or branched C₁₋₂₀alkyl, optionally containing unsaturation, that is not substituted with a hydrogen bond donor;

with a benzo(crown-ether) having a formula (IV):

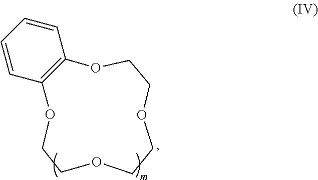

m being an integer from 1 to 3;

in the presence of an acylation acid catalyst to obtain a monoacylated benzo(crown-ether) having the formula (IA) depicted and described above.

In various aspects of the present disclosure, methods of forming an ion channel in a biological membrane are provided. The methods include combining the membrane with monoacylated benzo(crown-ether) (MAcBCE) compounds, monoalkylated benzo(crown-ether) (MAkBCE) compounds, or a combination thereof, such that the MAcBCE compounds, the MAkBCE compounds, or a combination of the MAcBCE and MAkBCE compounds self-assemble to form the ion channel in the membrane.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a plot of minimum inhibitory concentrations for various MAcBCE and MAkBCE compounds for the gram-positive bacteria Bacillus subtilis;

FIG. 2 a plot showing the normalized time course of resofurin fluorescence caused by the release of the cytoplasmic enzyme lactate dehydrogenase from B. subtilis cells in response to treatment with various bioactive agents;

FIG. 3 is a plot showing normalized resofurin fluorescence at the end of the treatment experiments for each of the bioactive agents shown in FIG. 2;

FIGS. 4 and 5 are plots showing the time course of DiSC3(5) fluorescence from B. subtilis cells in response to the addition of a MAkBCE compound and MAcBCE compound, respectively;

FIGS. 6-8 are plots showing average final fluorescence values of DiSC3(5) from B. subtilis cells in response to the addition of various MAkBCE compounds;

FIGS. 9-11 are plots showing average final fluorescence values of DiSC3(5) from B. subtilis cells in response to the addition of various MAcBCE compounds;

FIGS. 12-14 show single channel activity elicited by a MAcBCE compound in the presence of symmetrical KCl, NaCl and NMDG-Cl solutions;

FIGS. 15-17 show single channel activity elicited by a MAkBCE compound in the presence of symmetrical KCl, NaCl and NMDG-Cl solutions;

FIG. 18 shows multiple voltage ramps (N=100) from +100 mV to −100 mV applied in the presence of a MAkBCE compound;

FIG. 19 shows the variance curve calculated from the experiment in FIG. 18;

FIG. 20 shows multiple voltage ramps (N=100) from +100 mV to −100 mV applied in the presence of a MAcBCE compound;

FIG. 21 shows the variance curve calculated from the experiment in FIG. 20;

FIG. 22 is a plot of minimum inhibitory concentrations for various dialkylated lariat ethers toward B. subtilis, E. coli, and HEK293T cells;

FIGS. 23-31 are plots the time course of DiSC₃(5) fluorescence due to the activity of dialkylated lariat ethers with alkyl chains 6 to 14 carbons in length;

FIG. 32 shows relative DiSC₃(5) release after 10 min of treatment with 2 μM of various dialkylated lariat ethers in the presence of 60 mM KCl or 60 mM NMDG-Cl;

FIG. 33 shows a time course of normalized changes in DiSC₃(5) fluorescence due to the activity of 2 μM valinomycin in the presence of KCl, NaCl or NMDG-Cl;

FIG. 34 shows fluorescence values at the end of the experiment of FIG. 33;

FIG. 35 shows a time course of normalized changes in DiSC₃(5) fluorescence due to the activity of 2 μM LEC₁₀ in the presence of KCl, NaCl or NMDG-Cl;

FIG. 36 shows fluorescence values at the end of the experiment of FIG. 35;

FIG. 37 shows a time course of the DiSC₃(5) fluorescence due to the activity of LEC₁₀, performed in the presence of KCl or a cation-free dextrose solution;

FIG. 38 shows relative DiSC₃(5) release after 10 min of treatment with 2 μM LEC₁₀ in the presence or the absence of KCl;

FIG. 39 shows the normalized time course of the resofurin fluorescence, demonstrating LDH release from B. subtilis in response to LEC₁₀;

FIG. 40 shows normalized resofurin fluorescence at the end of the experiment as shown in FIG. 39; and

FIGS. 41 and 42 lipid bilayer recordings in response to current applied through a asolectin planar lipid bilayer clamped at 100 mV, recorded in the presence of 2 μM gramicidin and 2 μM LEC₁₀, respectively.

### DETAILED DESCRIPTION OF THE INVENTION

In various aspects, the synthesis and characterization of two new families of benzo(crown-ether) compounds, termed monoacylated benzo(crown-ether) (MAcBCE) compounds and monoalkylated benzo(crown-ether) (MAkBCE) compounds, are disclosed. The MAcBCE compounds and the MAkBCE compounds can self-assemble into non-toxic, membrane-stable supramolecular ion channels on biological membranes. Unlike other benzo(crown-ethers), the MAcBCE and MAkBCE compounds disclosed herein do not cause membrane lysis at high concentration (in the micromolar range), and thereby are suitable building blocks for bio-compatible synthetic ion channels. The MAcBCE and MAkBCE compounds may also be suitable for use in drug delivery systems, for example, by acting as sensors in nanoparticles or liposomes.

In various aspects, the MAcBCE compounds and the MAkBCE compounds disclosed herein include a benzo(crown-ether) that is monosubstituted with an acyl group or an alkyl group, respectively, that is not substituted with hydrogen bond donors. Synthetic ion channels based on benzo(crown-ether) compounds have been previously reported to function as ion selective channels in planar lipid bilayers, presumably by forming self-aggregated complexes via hydrogen-bonding networks. In contrast to previous versions of benzo(crown-ethers), the MAcBCE and MAkBCE compounds disclosed herein do not include a ureido group previously implicated as essential to H-bonding and columnar assembly. While hydrogen bonding networks previously implicated in the formation of supramolecular columnar structures were thought to be essential for channel activity, the presently disclosed MAcBCE and MAkBCE compounds that lack the ability to engage in H-bonding display robust channel activity.

The disclosed MAcBCE and MAkBCE compounds form scaffolds that exhibit ion channel activity in both biological and synthetic lipid bilayers. The MAcBCE and MAkBCE-assembled channels show some degree of ion preference, for example, a slight preference for transporting potassium cations (K⁺) over sodium cations (Na⁺) and N-methyl-d-glucamine cations (NMDG⁺). The current generation of ion channels is nonetheless permeable to different sized cations. As described in the examples below, single-channel recordings reveal that the probability of channel formation is higher in the presence of K+ as compared to Na⁺. Upon channel opening, the channels are not highly selective for the various cations. Without being limited to any particular theory, the ionic preference of benzo(crown-ether) compounds is likely due to the regulation of assembly by permeant ions, rather than ion-selective transport through crown-ether scaffolds.

### Definitions

The following includes a description of a number of terms, abbreviations or other shorthand as used herein, unless otherwise indicated. Any term, abbreviation or shorthand not explicitly described is understood to have the ordinary meaning used by a person of ordinary skill in the art.

The term “anion”, as used herein, unless otherwise indicated, means a negatively-charged ion.

The term “cation”, as used herein, unless otherwise indicated, means a positively-charged ion.

The terms “biological membrane”, “bilayer membrane” or “lipid bilayer”, as used herein, unless otherwise indicated, refer to a bimolecular assembly that forms a permeability barrier surrounding cells, intracellular compartments, liposomes, and other organelles. The membrane may include any of a large number of amphipathic lipid molecules but in cells it is primarily comprised of phospholipids.

The term “cell”, as used herein, unless otherwise indicated, refers to prokaryotic cell, yeast cell, eukaryotic cell, plant cell, human cell, or an animal cell.

The term “membrane”, as used herein, unless otherwise indicated, refers to a semi-permeable barrier that separates two liquid phases which may have the same or different compositions.

The term “cell membrane”, as used herein, unless otherwise indicated, refers to a selectively permeable lipid bilayer coated by proteins. The cell membrane comprises the outer layer of a cell.

The terms “channel” or “ion channel”, as used herein, unless otherwise indicated, refer to an aqueous diffusion pathway for membrane impermeant compounds usually formed by a pore within a cell membrane permitting the transfer of neutral or ionic species through it from one side of the membrane to the other.

The term “supramolecular assembly”, as used herein, unless otherwise indicated, refers to a complex of molecules or compounds held together by noncovalent bonds such as van de Waals force or hydrogen bonds. A supramolecular assembly can comprise two or more molecules or compounds.

The supramolecular assembly can be in any form or shape such as sphere, cylinder, disk, or sheet which can be solid or hollow. In some embodiments, the supramolecular assembly is in the form of a channel with a pore. The dimensions of supramolecular assemblies can range from nanometers to micrometers.

The term “self-assembly”, as used herein, unless otherwise indicated, refers to the assembly of molecules or compounds to form supramolecular assemblies without guidance or management from an outside source.

The term “self-assembling compound” or “self-assembling molecule”, as used herein, unless otherwise indicated, refer to the compound or molecule that can form a supramolecular assembly through a self-assembly process.

The term “hydrogen bond acceptor”, as used herein, unless otherwise indicated, refers to a molecule or group comprising a highly electronegative atom such as a nitrogen, oxygen, sulfur, fluorine, chlorine, and bromine, for example, the electronegative atom being susceptible to attract by means of electrostatic field a hydrogen atom nearby, thus forming a hydrogen bond with a hydrogen bond donor group. Non-limiting examples of hydrogen bond acceptors are groups comprising nitrogen or oxygen atoms with non-bonding doublets such as ether, ester, ketone and amide groups, or non-quaternized amine groups.

The term “hydrogen bond donor”, as used herein, unless otherwise indicated, refers to a molecule or group comprising an atom, or a group of atoms, wherein a hydrogen atom is covalently bound to a highly electronegative atom such as nitrogen, oxygen, sulfur or fluorine atom, the hydrogen atom being susceptible to be attracted by the electrostatic field of another highly electronegative atom nearby, thus forming a hydrogen bond with a hydrogen bond acceptor group. Non-limiting examples of hydrogen bond donors are groups such as hydroxyl groups, primary and secondary amine groups, carboxylic acids, and primary and secondary amide groups.

The term “liposome”, as used herein, unless otherwise indicated, refers to an artificial sac, usually spherical, consisting of one or more bilayer membranes of phospholipid that encloses an aqueous core and may mimic biological membranes.

The term “selectivity”, as used herein, unless otherwise indicated, refers to a measurable preference for one species over another, including cation over anion, anion over cation, one cation over a different cation, or one anion over a different anion.

The term “transport”, as used herein, unless otherwise indicated, refers to the movement of an ion or other species across a membrane boundary.

The term “ionophore”, as used herein, unless otherwise indicated, refers to a molecule, compound, supramolecular assembly, or other chemical species that facilitates the transport of ions across a biological membrane. The ionophore may be a lipid-soluble entity and may reversibly bind ions.

The term “integer”, as used herein, unless otherwise indicated, refers to a whole number that can be written without a fractional component or a remainder, including 0.

The terms “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl.

An alkyl can be saturated, or may “contain unsaturation”, meaning the alkyl is partially saturated or unsaturated. The alkyl can be optionally substituted.

The term “carboxyl”, as used herein, unless otherwise indicated, can include a functional group consisting of a carbon atom double-bonded to an oxygen atom and single-bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate”, as used herein, is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “substituted”, as used herein, refers to a compound or chemical moiety in which at least one hydrogen atom or at least one carbon atom of that compound or chemical moiety is replaced with a second chemical moiety. The second chemical moiety can be any desired substituent that does not adversely affect the desired activity of the compound.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “NMR”, as used herein, is intended to mean nuclear magnetic resonance. The term “HPLC”, as used herein, is intended to mean high-performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

Self-Assembling Compounds for Forming Ion Channels

Examples of self-assembling, ion channel forming agents are described herein. Self-assembling ion channel forming agents include monoacylated benzo(crown-ether) (MAcBCE) compounds and monoalkylated benzo(crown-ether) (MAkBCE) compounds.

The MAcBCE compounds may have the general formula (IA):

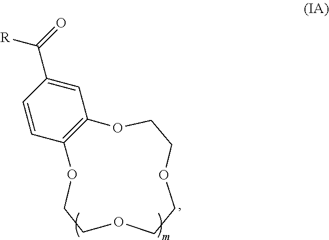

and the MAkBCE compounds may have the general formula (IIA):

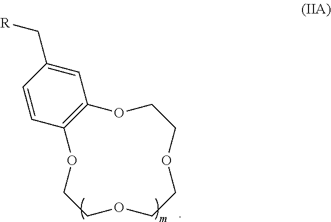

In each of the general formulas (IA) and (IIA), m is an integer from 1 to 3, and R may be straight chain or branched C₁₋₂₀alkyl, optionally containing unsaturation; a C₂₋₁₀cycloalkyl optionally containing unsaturation; straight chain or branched, saturated C₁₋₁₀alkyl; or an aryl comprising a phenyl. Any of the above can be further optionally substituted, with the proviso that R is not substituted with a hydrogen bond donor.

In some embodiments, in each of the general formulas (IA) and (IIA), m is an integer from 1 to 3 and R is a straight chain or branched C₁₋₂₀alkyl, optionally containing unsaturation, that is not substituted with a hydrogen bond donor.

In some embodiments, in each of the general formulas (IA) and (IIA), m is an integer from 1 to 3 and R is a straight chain, saturated C₁₋₂₀alkyl that is not substituted with a hydrogen bond donor.

In some embodiments, the MAcBCE compound has a formula (IB):

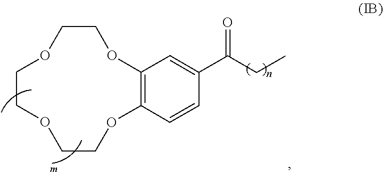

m being an integer from 1 to 3, and

n being an integer from 0 to 19, such as from 2 to 9, or n is selected from the group consisting of 2, 4, 6, 8, and 9.

In some embodiments, the MAkBCE compound has a formula (IIB):

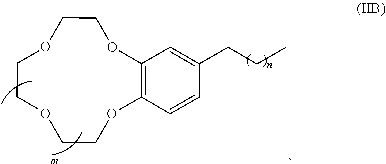

m being an integer from 1 to 3, and

n being an integer from 0 to 19, such as from 2 to 9, or n is selected from the group consisting of 2, 4, 6, 8, and 9.

The MAcBCE and MAkBCE compounds described herein can self-assemble into supramolecular ion channels in biological membranes. Without being bound by any theory, although the detailed mechanism is not specifically known, the self-assembling compounds disclosed herein are thought to form stacked arrays, where the macrocycles are oriented parallel to the membrane to allow sequential passage of ions. Alternative mechanisms are possible. The MAcBCE and MAkBCE compounds, which lack the ability to form H-bonding networks, are shown to have toxicity on gram-positive bacteria and ion-specific depolarizing activity of cell membranes. Moreover, after treatment with the MAcBCE and MAkBCE compounds, the lipid bilayer remains intact, and discrete changes in the conductance in the planar lipid bilayers are observed. These characteristics, which are described in more detail herein, demonstrate that the MAcBCE and MAkBCE compounds are ionophores that have ion channel activity in biological membranes.

Methods of Preparing Self-Assembling Compounds

The self-assembling benzo(crown-ether) compounds that are monosubstituted with one of an acyl group and an alkyl group are prepared by methods disclosed herein. An example method of preparing these monosubstituted benzo(crown-ether) compounds includes reacting a carboxylic acid having a formula (III):

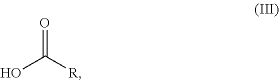

with a benzo(crown-ether) having a formula (IV):

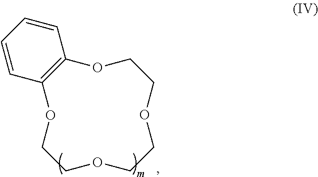

in the presence of an acylation acid catalyst to obtain a monoacylated benzo(crown-ether) having the formula (IA) or (IB) as described above.

The R group of the formula (III) is suitably the same as the R groups described above for the formulas (IA) and (IIA). For example, R may be a straight chain or branched C₁₋₂₀alkyl, optionally containing unsaturation, that is not substituted with a hydrogen bond donor. In some embodiments, R is a straight chain, saturated C₁₋₂₀alkyl that is not substituted with a hydrogen bond donor.

In some embodiments, the R group of the formula (III) is a straight chain, saturated alkyl group suitable to obtain the monosubstituted benzo(crown-ether) compounds having a formula (IB) or (IIB). For example, R may be a straight chain, saturated C₁₋₂₀alkyl, or a C₃₋₁₀alkyl, that is not substituted with a hydrogen bond donor. In some embodiments, R is a straight chain, saturated C₃, C₅, C₇, C₉, or C₁₀ alkyl that is not substituted with a hydrogen bond donor.

In the formula (IV), similar to formulas (IA), (IB), (IIA), and (IIB), m is an integer from 1 to 3.

Suitable acylation acid catalysts include, for example, Eaton's reagent (a solution of 10% wt/wt phosphorus pentoxide in methanesulfonic acid), and polyphosphoric acid (PPA). In one particular example, Eaton's reagent is used as the acylation acid catalyst. In other examples, the carboxylic acid having the formula (III) may be reacted with the benzo(crown-ether) having the formula (IV) in the presence of a mixture of carboxylic acids and anhydrides and PPA, or in the presence of sodium acetate and PPA, to promote an acylation reaction.

Suitable benzo(crown-ether) reagents utilized to obtain the monosubstituted benzo(crown-ethers) described herein are commercially available, for example, from Tokyo Chemical Industry (TCI), Tokyo, Japan.

The reaction between the benzo(crown-ether) and the carboxylic acid in the presence of the acylation acid catalyst is performed at a suitable temperature and suitable duration to ensure the reaction is carried out to completion with little to no loss in yield. As the reaction progresses, the reaction mixture may eventually turn red (e.g., a dark red color or bright cherry red color) which indicates that the reaction is unlikely to undergo further conversion to product. In some embodiments, the reaction may be performed at a temperature from 10° C. to 100° C. A suitable duration of the reaction may depend on the temperature at which the reaction is performed. For example, where the reaction between the benzo(crown-ether) and the carboxylic acid is carried out at or near room temperature (e.g., at a temperature from 10° C. to 30° C., or around 20° C.), the reaction may be carried out for a duration of from 4 hours to 6 hours until the reaction mixture turns the indicative red color. In other examples, the reaction may be carried out at an elevated temperature from 40° C. to 100° C., such as from 50° C. to 90° C., from 50° C. to 60° C., or from 60° C. to 90° C., and a duration of less than or equal to 1 hour. Where the reaction is carried out at these elevated temperatures, fewer byproducts may be formed. Moreover, the heated reaction mixture may be quenched with cold water at a suitable time after the red color (e.g., bright cherry red color) is observed. Suitably, the heated reaction mixture may be quenched within a time window of 20 minutes to 30 minutes after the red color is observed to ensure little to no loss in yield and/or to facilitate minimizing or reducing byproduct formation.

To obtain a monoalkylated benzo(crown-ether) having the formula (IIA) or (IIB) as described above, the monoacylated benzo(crown-ether) having the formula (IA) or (IB), respectively, obtained as described above can be reduced in the presence of a reducing agent. For example, the reducing agent may be a hydrosilane, such as triethyl silane (Et₃SiH), in which case a hydrogenation acid may be used, such as trifluoracetic acid (CF₃COOH), for example. The reduction reaction may be performed in an inert atmosphere or air, at a temperature at or near room temperature (e.g., from 10° C. to 30° C.), and for a suitable duration, for example, of about 3 hours. In another example, the monoacylated benzo(crown-ether) may be reduced in a solution with ethanol in the presence of hydrogen (H₂) and a palladium on carbon (Pd/C) catalyst.

Formulation

The self-assembling, ion channel forming agents described herein, and compositions including these agents, can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a self-assembling, ion channel forming, biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided are methods of forming an ion channel in a biological membrane. Example methods include combining a biological membrane with monoacylated benzo(crown-ether) (MAcBCE) compounds, monoalkylated benzo(crown-ether) (MAkBCE) compounds, or a combination thereof. The MAcBCE compounds, the MAkBCE compounds, or a combination of the MAcBCE and MAkBCE compounds, when combined with the biological membrane, self-assemble to form the ion channel in the membrane. The ion channels formed in the membrane by the self-assembling MAcBCE and MAkBCE compounds suitably facilitate transport of cationic species across the membrane. In some embodiments, the ion channel is a potassium (K⁺) or a sodium (Na⁺) cation channel. In some embodiments, the ion channels formed in the membrane by the MAcBCE and MAkBCE compounds may not display selectivity between cation species, such as between K⁺ and Na⁺ cations.

Also provided are methods of treating, preventing, or reversing a gram-positive bacteria infection or an excitability disorder in a subject in need thereof. Example methods includes administering a therapeutically effective amount of at least one MAcBCE compound, at least one MAkBCE compound, or a combination thereof, to a subject, so as to form at least one ion channel in a biological membrane. The at least one MAcBCE compound, at least one MAkBCE combination, or the combination thereof, may be administered so as to form the at least one ion channel in vivo. In some aspects, at least one MAcBCE compound, at least one MAkBCE combination, or the combination thereof, may act as an antibiotic against gram-positive bacteria including, but not limited to, Bacillus subtilis. In other aspects, the MAcBCE and MAkBCE compounds may be used, individually or in combination, as a treatment for an excitability disorder.

The MAcBCE and MAkBCE compounds utilized in the methods described herein are of the same composition as described above. For example, the MAcBCE compounds may have a formula (IA) or (IB), as described above. The MAkBCE compounds may have a formula (IIA) or (IIB), as described above. Moreover, the MAcBCE and MAkBCE may be prepared according to the methods of preparing described herein.

Methods described herein are generally performed on a subject in need thereof. A subject in need of the therapeutic methods described herein can be a subject having, diagnosed with, suspected of having, or at risk for developing a gram-positive bacteria infection or an excitability disorder. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of at least one monoacylated benzo(crown-ether) (MAcBCE) compound, at least one monoalkylated benzo(crown-ether) (MAkBCE) compound, or a combination thereof, is, for example, an amount that would cause the desired therapeutic effect in a subject while minimizing undesired side effects. In various embodiments, an effective amount of at least one MAcBCE compound and at least one MAkBCE compound, administered individually or in combination, can substantially inhibit, slow the progress of, or limit the development of a gram-positive bacteria infection or an excitability disorder.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of at least one at least one MAcBCE compound and at least one MAkBCE compound, administered individually or in combination, can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a gram-positive bacteria infection or an excitability disorder.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of at least one at least one MAcBCE compound and at least one MAkBCE compound, individually or in combination, can occur as a single event or over a time course of treatment. For example, at least one MAcBCE compound and at least one MAkBCE compound, individually or in combination, can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities.

A MAcBCE compound and a MAkBCE compound, individually or in combination, can be administered simultaneously or sequentially with another agent, such as an antibiotic, an anti-inflammatory, or another agent. For example, a MAcBCE compound and a MAkBCE compound, individually or in combination, can be administered simultaneously with another agent, such as an antibiotic or an anti-inflammatory. Simultaneous administration can occur through the administration of separate compositions, each containing one or more of a MAcBCE compound, a MAkBCE compound, an antibiotic, an anti-inflammatory, or another agent. Simultaneous administration can occur through the administration of one composition containing two or more of a MAcBCE compound, a MAkBCE compound, an antibiotic, an anti-inflammatory, or another agent. At least one MAcBCE compound and at least one MAkBCE compound, individually or in combination, can be administered sequentially with an antibiotic, an anti-inflammatory, or another agent. For example, at least one MAcBCE compound and at least one MAkBCE compound, individually or in combination, can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve the taste of the product; or improve the shelf life of the product.

Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to at least one monoacylated benzo(crown-ether) (MAcBCE) compound, at least one monoalkylated benzo(crown-ether) (MAkBCE) compound, a combination thereof, or formulations including one of or both of these compounds. The MAcBCE compounds may have a formula (IA) or (IB) as described above, and the MAkBCE compounds may have a formula (IIA) or (IIB) as described above. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects, or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### A. Re-Evaluation of the Mechanism of Cytotoxicity of Dialkylated Lariat Ether Compounds

Crown ethers and their derivatives have generated wide interest due to their ability to form stable complexes with cations. These properties have been successfully exploited in ion transport through bulk liquid membranes as well as in sensors and scaffolds for materials, developments which prompted their examination as biologically relevant ionophores. The transport of ions through biological membranes underlies many key physiological processes and understanding the complexities of this phenomenon continues to be an area of active research. Crown ethers are potentially powerful tools in this pursuit, due to their binding properties and highly customizable structures. Indeed, crown ether derivatives, such as monoalkylated and dialkylated lariat ethers, amphiphilic benzo(crown-ether) derivatives, hydraphiles, and ion shuttles have been demonstrated to function as ionophores. Despite these successful examples, understanding of the mechanisms of ion transport by these crown ether derivatives remains limited. An improved understanding of these processes will provide critical insights that will both advance fundamental knowledge about ion transport mechanisms and provide a framework for the rational design of synthetic ionophores with well-defined properties.

Disclosed herein is an in-depth evaluation of various dialkylated diaza(18-crown-6) ethers, which are a subset of the lariat class of crown ethers. Lariat ethers are macrocyclic crown ethers with one or more sidearms attached to the macrocyclic core structure. Lariat ethers have been reported to bind alkali cations and behave as ionophores in bulk liquid membranes and ion-selective electrodes. Dialkylated diaza(18-crown-6) ethers have been reported to have toxic activity towards prokaryotic and eukaryotic cells, and evidence from toxicity and depolarization assays initially suggested that these compounds behave as ion carriers. See Leevy, W. M. et al., Correlation of bilayer membrane cation transport and biological activity in alkyl-substituted lariat ethers, Org. Biomol. Chem., 2005, 3, 1647-1652. However, experiments in asolectin bilayers revealed that dioctylated and diundecylated lariat ethers elicit discrete increases in membrane conductance, a result typical of ion channels, as opposed to ion carriers. See Negin, S. et al., Antibiotic Potency against E. coli Is Enhanced by Channel-Forming Alkyl Lariat Ethers, Chembiochem, 2016, 17, 2153-2161. Moreover, the effect of the alkyl chain lengths on the toxicity and transport implied that the interaction of these compounds with a bilayer membrane differs from their behavior in a bulk liquid membrane. In general, the literature suggests that hydrophobic lariat ethers that bear longer alkyl chains function as more efficient cation carriers. In contrast, dialkylated lariat ethers show peak activity when a 10 carbon chain is present on the core, with the activity diminishing with increasing chain length. This observation prompted Leevy et al. to propose that dialkylated lariat ethers require a minimum hydrophobicity to act as ion carriers, but when the alkyl chains are too long, the molecules are able to nest inertly within the membrane.

In order to test this mechanism and establish a deeper understanding of their transport behavior in membranes, a representative set of diaza(18-crown-6) ethers bearing dialkylated tails ranging from 6 to 14 carbons are studied.

Synthesis and Preparation of Dialkylated Diaza(18-Crown-6) Ether Compounds

The series of symmetrical dialkylated diaza(18-crown-6) ethers were prepared according to the following general procedure. A simple one-step reductive amination of 4,13-diaza(18-crown-6) and the appropriate aldehyde is utilized, as opposed to previous two-step procedures.

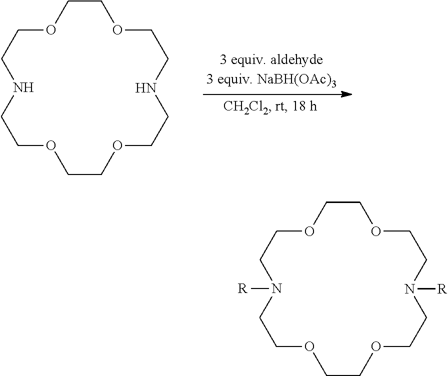

In the procedure shown above, R is a straight chain, saturated C₆₋₁₄alkyl.

All glassware was either oven dried at 140° C. or flame dried under vacuum and purged with nitrogen immediately prior to use. Hexanal, heptanal, octanal, nonanal, decanal, undecanal, dodecanal, tridecanol, and pyridinium chlorochromate (PCC) were obtained from Millipore Sigma. Unless otherwise specified, reagents were used as obtained from the supplier without further purification. Acetonitrile (MeCN), toluene, and dichloromethane (CH₂Cl₂) were freshly distilled from calcium hydride or passed through an alumina column immediately prior to use. Other solvents were purified using accepted procedures from the sixth edition of “Purification of Laboratory Chemicals”. Air- and moisture-sensitive reactions were performed using standard Schlenk techniques under an inert nitrogen atmosphere, unless otherwise specified. Analytical thin layer chromatography (TLC) was performed using pre-coated silica gel 60 F24 plates containing a fluorescent indicator. Reaction products were visualized using 254 nm UV light and ceric ammonium molybdate (CAM), KMnO₄, and I₂ stains unless otherwise specified. Preparative chromatography using a gradient method with mixtures of MeOH and CH₂Cl₂ or EtOAc and hexanes, unless otherwise specified, was performed using SilicaFlash P60 silica gel (230-400 mesh) via Still's method.

In the example dialkylated lariat ethers described further below, each dialkylated diaza(18-crown-6) ether is identified with the code “LECₙ”, which refers to the dialkylated lariat ether substituted with an alkyl chains of n carbons.

Example dialkylated diaza(18-crown-6) ether compounds will now be described. The structures were confirmed by nuclear magnetic resonance imaging (NMR) and mass spectrometry (MS). ¹H NMR and ¹³C NMR spectra were obtained using Bruker Avance-500 spectrometers. Chemical shifts are reported relative to the tetramethylsilane peak (δ0.00 ppm). Accurate mass measurements were acquired at the University of Wisconsin, Madison, using a Micromass LCT (electrospray ionization or electron impact methods). Didecyl diaza(18-crown-6) (LEC₁₀) is the only lariat ether that is commercially available (Kryptofix® 22DD; Sigma-Aldrich); the effects of the purchased material were indistinguishable from the synthesized compound.

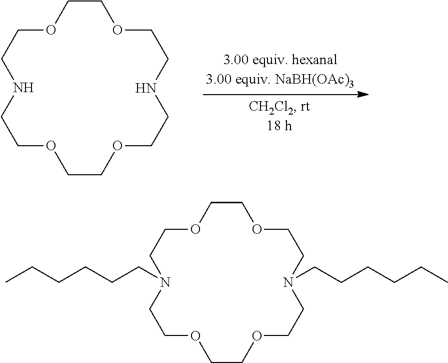 [SMILES: C1COCCOCCNCCOCCOCCN1 ; CCCCCCN1CCOCCOCCN(CCCCCC)CCOCCOCC1]

Dihexyl diaza(18-crown-6) (LEC₆). To a stirred solution of 100.0 mg (0.38 mmol, 1.00 equiv.) 1,4,10,13-tetraoxa-7,16-diazacyclooctadecane in 3.80 mL CH₂Cl₂ was added 0.14 mL (1.14 mmol, 3.00 equiv.) hexanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₆. The crude material was purified via flash column chromatography on alumina using a 025% gradient of EtOAc in hexanes to afford 95.0 mg (0.22 mmol, 58%) of LEC₆ as a white solid. ¹H NMR (500 MHz, CDCl₃) δ 3.71-3.51 (m, 16H), 2.85-2.69 (m, 8H), 2.58-2.41 (m, 4H), 1.51-1.37 (m, 4H), 1.35-1.20 (m, 12H), 0.88 (t, J=6.9 Hz, 6H). ¹³C NMR (126 MHz, CDCl₃) δ 70.8, 70.1, 56.1, 53.9, 31.8, 27.2, 22.7, 14.1. HRMS (ESI) m/z calculated for C₂₄H₅₀N₂O₄ [M+H]⁺ 431.3843, found 431.3843.

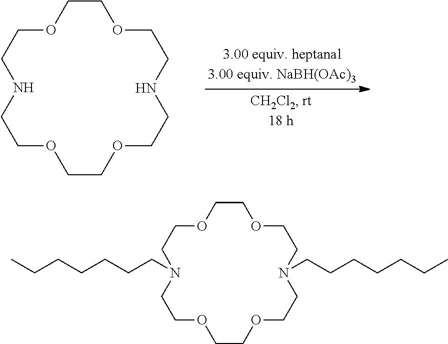 [SMILES: C1COCCOCCNCCOCCOCCN1 ; CCCCCCCN1CCOCCOCCN(CCCCCCC)CCOCCOCC1]

Diheptyl diaza(18-crown-6) (LEC₇). To a stirred solution of 100.0 mg (0.38 mmol, 1.00 equiv.) 1,4,10,13-tetraoxa-7,16-diazacyclooctadecane in 3.80 mL CH₂Cl₂ was added 0.16 mL (1.14 mmol, 3.00 equiv.) heptanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₇. The crude material was purified via flash column chromatography on alumina using a 0-25% gradient of EtOAc in hexanes to afford 49.0 mg (0.11 mmol, 29%) of LEC₇ as a white solid. ¹H NMR (500 MHz, CDCl₃) δ 3.64-3.56 (m, 16H), 2.77 (t, J=6.0 Hz, 8H), 2.51-2.44 (m, 4H), 1.49-1.39 (m, 4H), 1.33-1.21 (m, 16H), 0.91-0.84 (m, 6H). ¹³C NMR (126 MHz, CDCl₃) δ 70.8, 70.8, 70.1, 70.1, 56.1, 54.0, 31.9, 29.3, 27.5, 27.3, 22.6, 14.1. HRMS (ESI) m/z calculated for C₂₆H₅₄N₂O₄ [M+H]⁺ 459.4156, found 459.4153.

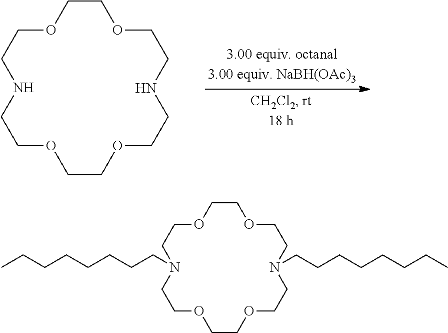 [SMILES: C1COCCOCCNCCOCCOCCN1 ; CCCCCCCCN1CCOCCOCCN(CCCCCCCC)CCOCCOCC1]

Dioctyl diaza(18-crown-6) (LEC₈). To a stirred solution of 100.0 mg (0.38 mmol, 1.00 equiv.) 1,4,10,13-tetraoxa-7,16-diazacyclooctadecane in 3.80 mL CH₂Cl₂ was added 0.18 mL (1.14 mmol, 3.00 equiv.) octanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₈. The crude material was purified via flash column chromatography on alumina using a 0-25% gradient of EtOAc in hexanes to afford 49.0 mg (0.10 mmol, 26%) of LEC₈ as a white solid. ¹H NMR (500 MHz, CDCl₃) δ 3.64-3.56 (m, 16H), 2.77 (t, J=6.0 Hz, 8H), 2.53-2.44 (m, 4H), 1.48-1.38 (m, 4H), 1.32-1.18 (m, 20H), 0.91-0.86 (m, 6H). ¹³C NMR (126 MHz, CDCl₃) δ 70.7, 69.8, 55.9, 53.8, 31.8, 29.7, 29.5, 29.4, 29.3, 27.5, 22.7, 22.7, 14.1. HRMS (ESI) m/z calculated for C₂₈H₅₈N₂O₄ [M+H]⁺ 487.4469, found 487.4471.

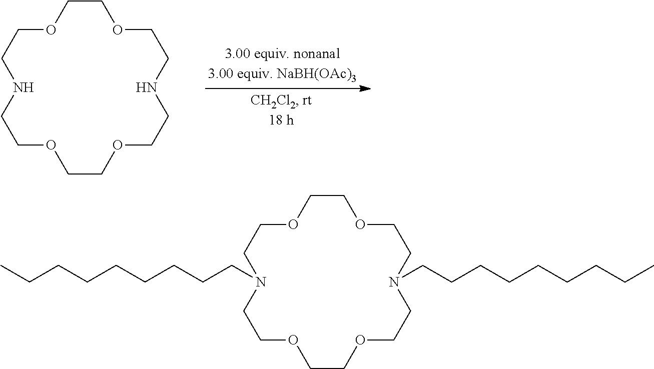 [SMILES: C1COCCOCCNCCOCCOCCN1 ; CCCCCCCCCN1CCOCCOCCN(CCCCCCCCC)CCOCCOCC1]

Dinonyl diaza(18-crown-6) (LEC₉). To a stirred solution of 100.0 mg (0.38 mmol, 1.00 equiv.) 1,4,10,13-tetraoxa-7,16-diazacyclooctadecane in 3.80 mL CH₂Cl₂ was added 0.20 mL (1.14 mmol, 3.00 equiv.) nonanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₉. The crude material was purified via flash column chromatography on alumina using a 0-25% gradient of EtOAc in hexanes to afford 66.0 mg (0.13 mmol, 34%) of LEC₉ as a white solid. ¹H NMR (500 MHz, CDCl₃) δ 3.61 (d, J=7.4 Hz, 16H), 2.77 (t, J=6.0 Hz, 8H), 2.52-2.43 (m, 4H), 1.48-1.38 (m, 4H), 1.26 (s, 24H), 0.88 (t, J=6.9 Hz, 6H). ¹³C NMR (126 MHz, CDCl₃) δ 70.8, 70.0, 56.1, 53.9, 31.9, 29.7, 29.6, 29.6, 29.3, 27.5, 27.3, 22.7, 14.1. HRMS (ESI) m/z calculated for C₃₀H₆₂N₂O₄ [M+H]⁺ 515.4782, found 515.4787.

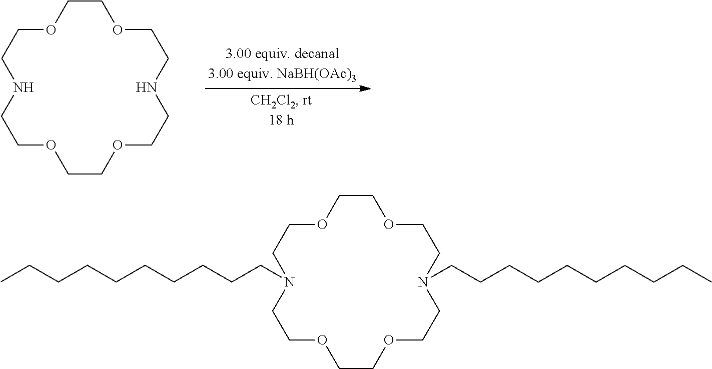 [SMILES: C1COCCOCCNCCOCCOCCN1 ; CCCCCCCCCCN1CCOCCOCCN(CCCCCCCCCC)CCOCCOCC1]

Didecyl diaza(18-crown-6) (LEC₁₀). To a stirred solution of 100.0 mg (0.38 mmol, 1.00 equiv.) 1,4,10,13-tetraoxa-7,16-diazacyclooctadecane in 3.80 mL CH₂Cl₂ was added 0.21 mL (1.14 mmol, 3.00 equiv.) decanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₁₀. The crude material was purified via flash column chromatography on alumina using a 0-25% gradient of EtOAc in hexanes to afford 19.0 mg (0.03 mmol, 8%) of LEC₁₀ as a white solid. ¹H NMR (500 MHz, CDCl₃) δ 3.61 (d, J=8.6 Hz, 16H), 2.77 (t, J=6.0 Hz, 8H), 2.52-2.43 (m, 4H), 1.43 (dq, J=13.3, 6.7, 6.0 Hz, 4H), 1.26 (s, 28H), 0.88 (t, J=6.9 Hz, 6H). ¹³C NMR (126 MHz, CDCl₃) δ 70.8, 70.1, 56.1, 54.0, 31.9, 29.7, 29.7, 29.6, 29.6, 29.3, 27.5, 27.3, 22.7, 14.1. HRMS (ESI) m/z calculated for C₃₂H₆₆N₂O₄ [M+H]⁺ 543.5095, found 543.5097.

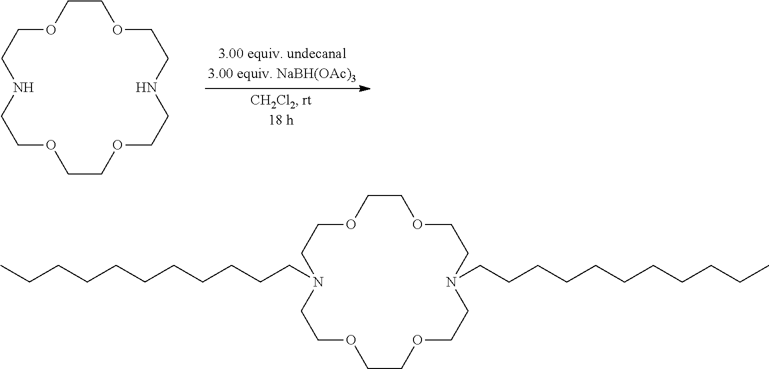 [SMILES: C1COCCOCCNCCOCCOCCN1 ; CCCCCCCCCCCN1CCOCCOCCN(CCCCCCCCCCC)CCOCCOCC1]

Diundecyl diaza(18-crown-6) (LEC₁₁). To a stirred solution of 100.0 mg (0.38 mmol, 1.00 equiv.) 1,4,10,13-tetraoxa-7,16-diazacyclooctadecane in 3.80 mL CH₂Cl₂ was added 0.24 mL (1.14 mmol, 3.00 equiv.) undecanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₁₁. The crude material was purified via flash column chromatography on alumina using a 0-25% gradient of EtOAc in hexanes to afford 119 mg (0.21 mmol, 55%) of LEC₁₁ as a white solid. ¹H NMR (500 MHz, CDCl₃) δ 3.76-3.47 (m, 16H), 2.90-2.64 (m, 8H), 2.57-2.41 (m, 4H), 1.51-1.37 (m, 4H), 1.34-1.18 (m, 32H), 0.88 (t, J=6.9 Hz, 6H). ¹³C NMR (126 MHz, CDCl₃) δ 70.7, 70.0, 63.1, 56.0, 53.9, 32.8, 31.9, 31.9, 29.6, 29.6, 29.4, 29.4, 27.5, 27.2, 25.7, 22.7, 14.1. HRMS (ESI) m/z calculated for C₃₄H₇₀N₂O₄ [M+H]⁺ 571.5408, found 571.5405.

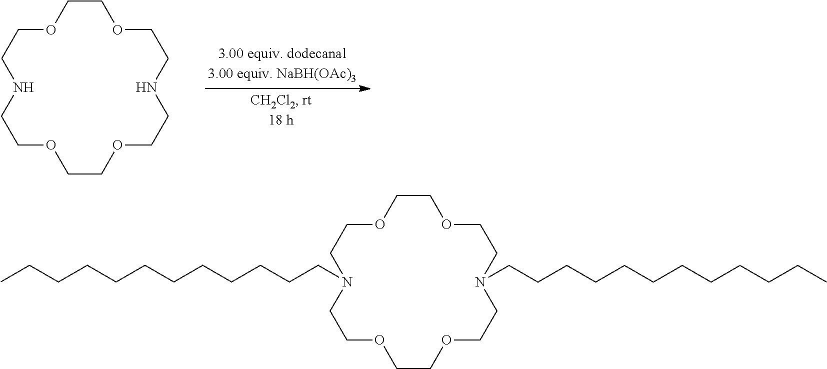 [SMILES: C1COCCOCCNCCOCCOCCN1 ; CCCCCCCCCCCCN1CCOCCOCCN(CCCCCCCCCCCC)CCOCCOCC1]

Didodecyl diaza(18-crown-6) (LEC₁₂). To a stirred solution of 100.0 mg (0.38 mmol, 1.00 equiv.) 1,4,10,13-tetraoxa-7,16-diazacyclooctadecane in 3.80 mL CH₂Cl₂ was added 0.25 mL (1.14 mmol, 3.00 equiv.) dodecanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₁₂. The crude material was purified via flash column chromatography on alumina using a 0-25% gradient of EtOAc in hexanes to afford 118 mg (0.20 mmol, 53%) of LEC₁₂ as a white solid. ¹H NMR (500 MHz, Chloroform-d) δ 3.61 (d, J=8.7 Hz, 16H), 2.77 (t, J=6.0 Hz, 8H), 2.51-2.44 (m, 4H), 1.76-1.69 (m, 2H), 1.43 (dq, J=12.9, 6.9, 6.5 Hz, 4H), 1.26 (s, 34H), 0.88 (t, J=6.9 Hz, 6H). ¹³C NMR (126 MHz, CDCl₃) δ 70.8, 70.1, 56.1, 54.0, 31.9, 29.7, 29.7, 29.6, 29.4, 27.5, 27.3, 22.7, 14.1. HRMS (ESI) m/z calculated for C₃₆H₇₄N₂O₄ [M+H]⁺ 599.5721, found 599.5716.

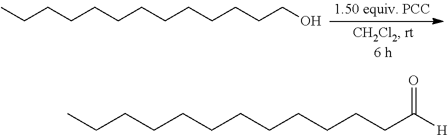

Tridecanal. To a stirred solution of 1.00 g (4.99 mmol, 1.00 equiv.) tridecanol in 50 mL CH₂Cl₂ was added 1.61 g (7.49 mmol, 1.50 equiv.) PCC. The resulting black solution was stirred at room temperature for 6 h, then 1.61 g celite was added and the light brown suspension stirred at room temperature for an additional 30 minutes. The crude reaction mixture was then filtered through a plug of silica gel using CH₂Cl₂ as eluent to afford 732 mg (3.69 mmol, 74%) tridecanal as a white solid which was used without any additional purification. ¹H NMR (500 MHz, CDCl₃) δ 9.76 (t, J=1.9 Hz, 1H), 2.41 (td, J=7.3, 1.9 Hz, 2H), 1.63 (p, J=7.4 Hz, 2H), 1.37-1.20 (m, 18H), 0.88 (t, J=6.9 Hz, 3H). ¹³C NMR (126 MHz, CDCl₃) δ 202.9, 43.9, 31.9, 29.6, 29.6, 29.6, 29.4, 29.4, 29.3, 29.2, 22.7, 22.1, 14.1. HRMS (ESI) m/z calculated for C₁₃H₂₆O [M+H]⁺ 199.2056, found 199.2055.

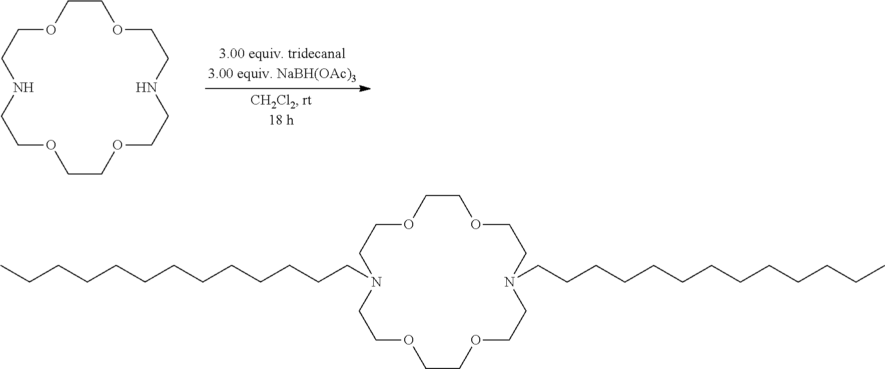 [SMILES: C1COCCOCCNCCOCCOCCN1 ; CCCCCCCCCCCCCN1CCOCCOCCN(CCCCCCCCCCCCC)CCOCCOCC1]

Ditridecyl diaza(18-crown-6) (LEC₁₃). To a stirred solution of 100.0 mg (0.38 mmol, 1.00 equiv.) 1,4,10,13-tetraoxa-7,16-diazacyclooctadecane in 3.80 mL CH₂Cl₂ was added 0.27 mL (1.14 mmol, 3.00 equiv.) tridecanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₁₃. The crude material was purified via flash column chromatography on alumina using a 0-25% gradient of EtOAc in hexanes to afford 58.0 mg (0.09 mmol, 24%) of LEC₁₃ as a white solid. ¹H NMR (500 MHz, CDCl₃) δ 3.66-3.55 (m, 16H), 2.77 (t, J=6.0 Hz, 8H), 2.51-2.45 (m, 4H), 1.78 (s, 2H), 1.43 (p, J=7.1 Hz, 4H), 1.25 (s, 38H), 0.88 (t, J=6.9 Hz, 6H). ¹³C NMR (126 MHz, CDCl₃) δ 70.8, 70.1, 56.1, 54.0, 31.9, 29.7, 29.7, 29.7, 29.6, 29.4, 27.5, 27.3, 22.7, 14.1. HRMS (ESI) m/z calculated for C₃₈H₇₈N₂O₄ [M+H]⁺ 627.6034, found 627.6021.

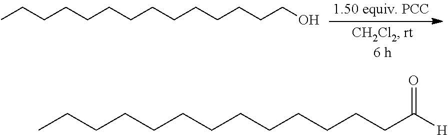

Tetradecanal. To a stirred solution of 1.00 g (4.66 mmol, 1.00 equiv.) tetradecanol in 47 mL CH₂Cl₂ was added 1.51 g (6.99 mmol, 1.50 equiv.) PCC. The resulting black solution was stirred at room temperature for 6 h, then 1.51 g celite was added and the light brown suspension stirred at room temperature for an additional 30 minutes. The crude reaction mixture was then filtered through a plug of silica gel using CH₂Cl₂ as eluent to afford 801 mg (3.77 mmol; 81%) tetradecanal as a white solid which was used without any additional purification. ¹H NMR (500 MHz, CDCl₃) δ 9.76 (t, J=1.9 Hz, 1H), 2.42 (td, J=7.3, 1.9 Hz, 2H), 1.63 (p, J=7.4 Hz, 2H), 1.37-1.19 (m, 20H), 0.88 (t, J=6.9 Hz, 3H). ¹³C NMR (126 MHz, CDCl₃) δ 202.9, 43.9, 31.9, 29.7, 29.6, 29.6, 29.4, 29.4, 29.2, 22.7, 22.1, 14.1. HRMS (ESI) m/z calculated for C₁₄H₂₈O [M+H]⁺ 213.2213, found 213.2210.

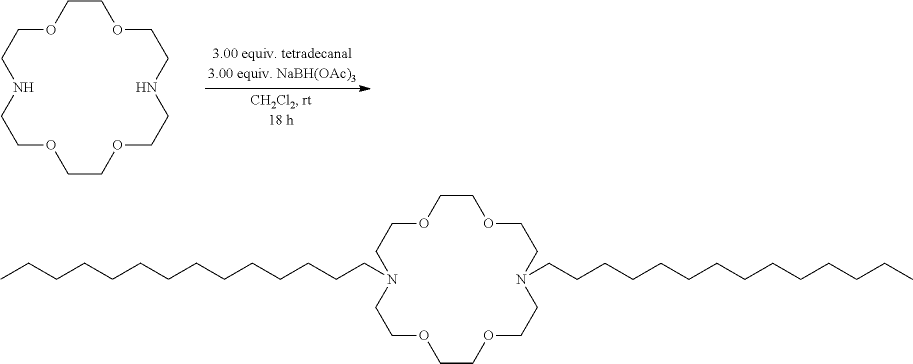 [SMILES: C1COCCOCCNCCOCCOCCN1 ; CCCCCCCCCCCCCCN1CCOCCOCCN(CCCCCCCCCCCCCC)CCOCCOCC1]

Ditetradecyl diaza(18-crown-6) (LEC₁₄). To a stirred solution of 100.0 mg (0.38 mmol, 1.00 equiv.) 1,4,10,13-tetraoxa-7,16-diazacyclooctadecane in 3.80 mL CH₂Cl₂ was added 242 mg (1.14 mmol, 3.00 equiv.) tetradecanal, followed by 242 mg (1.14 mmol, 3.00 equiv.) NaBH(OAc)₃. The white suspension was stirred at room temperature for 18 h, then filtered through a pad of celite. The filter pad was washed with additional CH₂Cl₂, and the volatiles removed in vacuo to afford crude LEC₁₄. The crude material was purified via flash column chromatography on alumina using a 0-25% gradient of EtOAc in hexanes to afford 78.0 mg (0.12 mmol, 32%) of LEC₁₄ as a white solid. ¹H NMR (500 MHz, CDCl₃) δ 3.67-3.53 (m, 16H), 2.77 (t, J=6.0 Hz, 8H), 2.52-2.42 (m, 4H), 1.58 (s, 8H), 1.43 (s, 4H), 1.25 (s, 36H), 0.88 (t, J=6.9 Hz, 6H). ¹³C NMR (126 MHz, CDCl₃) δ 70.8, 70.1, 56.1, 54.0, 31.9, 29.7, 29.7, 29.7, 29.4, 27.5, 27.3, 22.7, 14.1. HRMS (ESI) m/z calculated for C₄₀H₈₂N₂O₄ [M+H]⁺ 655.6347, found 655.6339.

### Experimental Results

The dialkylated lariat ethers were prepared as described above. Testing was conducted to evaluate the behavior of these compounds as ionophores for cell membranes. Specifically, the cyto-toxicity, ability to depolarize a membrane, planar lipid bilayer ion channel activity, and membrane lysis effect, of the dialkylated lariat ethers were evaluated. These results are described in further detail below. The acute release of lactate dehydrogenase, a complete lack of ion specificity, depolarization in the absence of extracellular ions, and a lack of discrete changes in the conductance in the planar lipid bilayers solidly demonstrates that biological activities of these dialkylated lariat ethers are due to their membrane lytic activity, as opposed to the expected ion transport activity.

Toxicity

The cyto-toxicity was determined by measuring the minimum inhibitory concentrations (MIC) for this series of dialkylated lariat ethers in the Gram-negative bacteria Escherichia coli, the Gram-positive bacteria Bacillus subtilis, and human embryonic kidney (HEK293T) cells.

On bacteria. XL1 blue strain Escherichia coli cells transformed with a pET28a plasmid (which contains a kanamycin resistance gene) were grown at 37° C. in 2 mL of LB medium (Miller's LB broth, Research Products International) supplemented with 50 μg/mL of kanamycin until the optical density (600 nm) reached 0.600. The starting density of bacteria in the remainder of the experiments was a 1/100 dilution of this bacterial density, which was split into 4 mL cultures. A series of 400, 100, 10 and 5 mM stock solutions of each dialkylated diaza(18-crown-6) compounds in trifluoroethanol (TFE) were diluted by adding to the 4 mL cultures until final concentrations of 400, 200, 100, 50, 20, 15, 10, 5, 4, 3, 2 and 1 μM were reached, with the final TFE concentration never >0.1% v/v. Negative controls in the presence of TFE 0.1% v/v or the absence of any treatment were done for each batch. The cultures were incubated at 37° C. with agitation for 12 h. Bacillus subtilis (168 WT) toxicity tests were carried out using an identical protocol, except for the addition of the antibiotic. For both bacterial species, the minimum inhibitory concentrations (MIC) were determined as the lowest compound concentration that inhibited growth after 12 h as judged by visual turbidity. Each compound was assayed a minimum of three times at each tested concentration.

On HEK293T cells. Human embryonic kidneys (HEK293T) cells (Thermofisher Scientific) were grown attached in cell culture dishes, detached by trypsin treatment and diluted in Dulbecco's Modified Eagle's Medium (DMEM) medium supplemented with phenol red indicator (Thermofisher scientific) until the density reached 100,000 viable cells/mL. An aliquot of 1 mL of this cell suspension was added to each well of a 24-wells culture plate. After 2 h, when the cells were already attached, a custom amount of 400, 100, 10 or 5 mM TFE solution of the dialkylated diaza(18-crown-6) ether compounds were added until final concentration of 400, 200, 100, 50, 20, 15, 10, 8, 6, 5, 4, 3, 2 or 1 μM were reached. The final TFE concentration never was over 0.1% v/v. Negative controls in the presence of TFE 0.1% v/v or the absence of any treatment were done for each plate. The plates were cultured at 37° C. The MIC was determined as the lowest compound concentration that killed all the cells after 48 h, as judged by trypan blue dye. The change in the pH of the medium, as judged by the change in the color (red to yellow) of the medium after 72 h, was also considered; this only occurred in those wells where the cells were still alive and multiplying. Each compound was assayed a minimum of three times at each reported concentration.

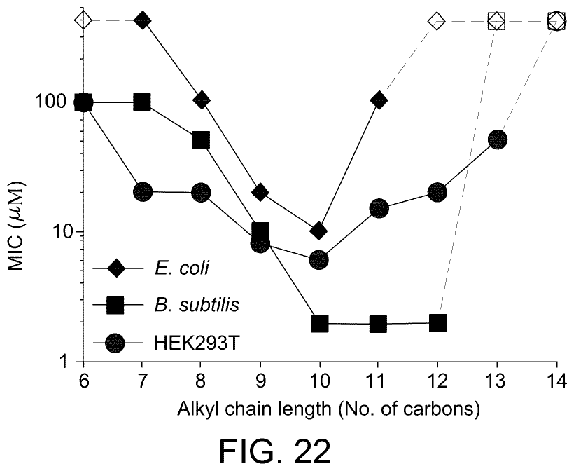

FIG. 22 shows the minimum inhibitory concentrations (MIC) towards B. subtilis, E. coli, and HEK293T cells. Open symbols represent non-determined values when the MIC is >400 μM. Each compound was assayed a minimum of three times at each different concentration. The results showed that B. subtilis is more susceptible to LEC₆-LEC₁₄, as judged by lower MICs; LEC₁₀ is the most toxic to E. coli (MIC=10 μM), while LEC₁₀, LEC₁₁, and LEC₁₂ are the most toxic to B. subtilis at concentrations as low as 2 μM (FIG. 22), results that are in good agreement with similar studies. Additionally, a remarkable discontinuity in the toxicity between LEC₁₂ and LEC₁₃ was observed, where the addition of only a single methylene group to the alkyl chain completely abrogates the toxicity towards B. subtilis from a MIC=2 μM with LEC₁₂ to undetectable with LEC₁₃ at concentration as high as 400 μM. The toxicities of dialkylated lariat ethers towards HEK293T cells were more consistent than the trends with E. coli and B. subtilis; however, LEC₁₀ (MIC=6 μM) proved to be the most toxic lariat ether towards all three tested cellular systems.

DiSC3(5) Depolarization Assays

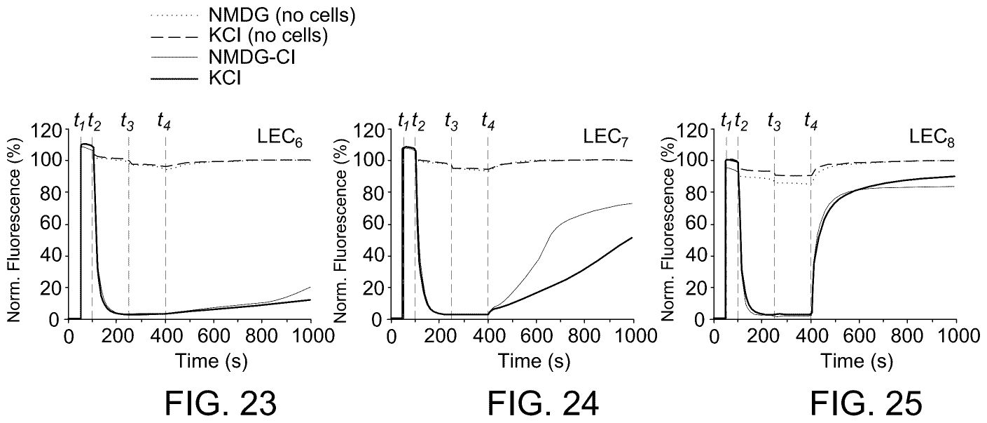

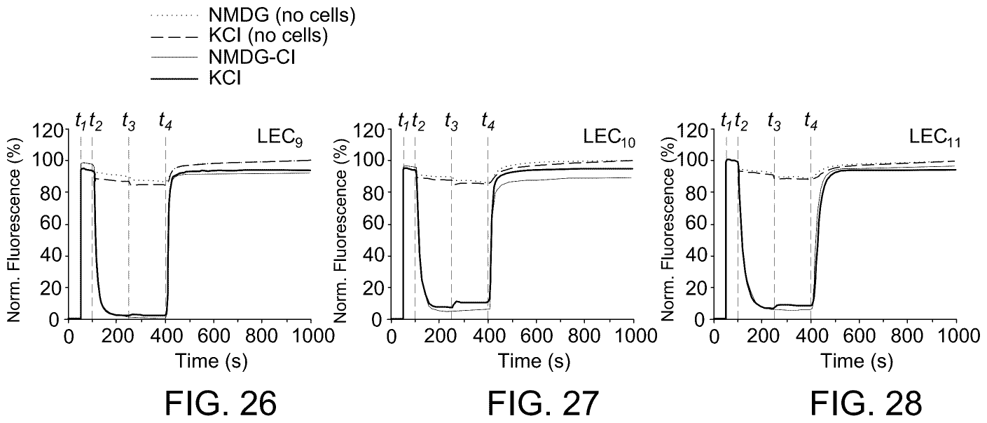

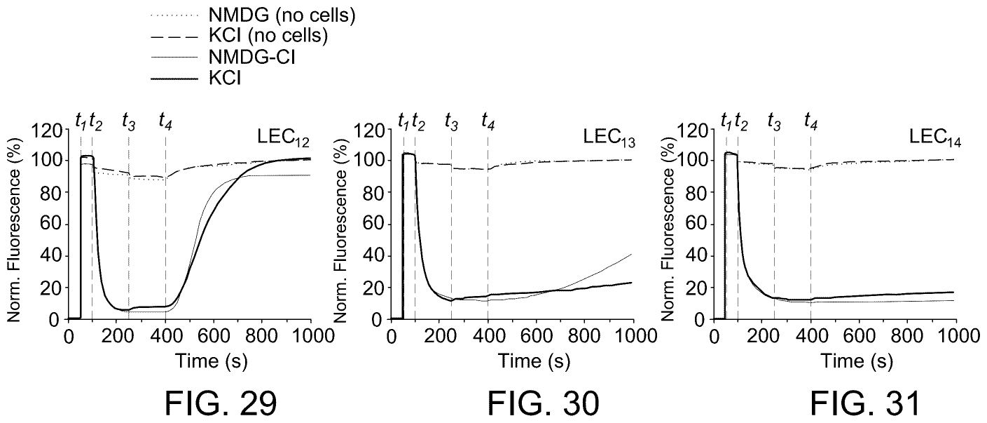

Dialkylated lariat ethers LEC₆-LEC₁₄ were then tested for their ability to depolarize a B. subtilis membrane using the fluorescent dye 3,3-dipropylthiadicarbocyanine (DiSC₃(5)), which undergoes membrane voltage-dependent partitioning between the intracellular and the extracellular medium. FIGS. 23-31 show the time course of the DiSC₃(5) fluorescence due to the activity of dialkylated lariat ethers with alkyl chains 6 to 14 carbons in length. The experiment was performed in a NMDG-MeSO₃ solution (100 mM NMDG, 10 mM HEPES, pH adjusted to 7.4 with methanesulfonic acid). The events are: t1, addition of dye; t2, addition of B. subtilis; t3, addition of a 2 M KCl or a 2 M NMDG-Cl solution up to a final concentration of 60 mM; t4, addition of dialkylated lariat ether up to 2 μM. Control experiments were performed that were similar to the two previously described experiments, with the exception that additional NMDG-MeSO₃ solution without cells was added at timepoint t2. The experiments were normalized relative to the final fluorescence intensity in the control experiments, as described below.

To conduct the experiments shown in FIGS. 23-31, a 2-mL liquid culture of B. subtilis cells was grown at 37° C. in LB media until OD₆₀₀=0.600, and then collected by centrifugation at 2000 rpm during 3 min. The bacteria were washed once in NMDG-MeSO₃ solution (100 mM NMDG, 10 mM HEPES, pH adjusted to 7.4 with methanesulfonic acid) or dextrose solution (200 mM dextrose, 10 mM Tris, pH adjusted to 7.4 with HCl) depending on the experiment. The centrifugation step was repeated and the bacteria were resuspended until OD₆₀₀=1.0 in the same solution. The working dye solution was a 200 μM solution of DiSC₃(5) (3,3′-dipropylthiadicarbocyanine iodide; Tokyo Chemical Industry) in DMSO. 2M solutions of NMDG-Cl, NaCl and KCl were prepared, they contained 10 mM HEPES and the pH was adjusted to pH 7.4 with NMDG, NaOH or KOH, respectively, to avoid any pH changes after their addition. The experiment was initiated with 3 mL of NMDG-MeSO₃ or dextrose solution in a quartz cuvette, then 5 μL of the dye solution was added (timepoint t1, 50 sec) for a 0.3 μM final concentration by the end of experiment. This was followed by the addition of 100 μL of the B. subtilis suspension (timepoint t2, 100 sec). After 150 sec (timepoint t3, 250 sec) the fluorescence stabilized to a minimum intensity and 100 μL of 2M NMDG-Cl, 2M NaCl or 2 M KCl solution was added to reach a about 60 mM final concentration of the salt. The final step was the addition 0.64 μL of a 10 mM stock solution of the desired dialkylated lariat ether compound in TFE (timepoint t4, 400 sec), for a final concentration of 2 μM. Negative controls were performed by adding the same volume of TFE without any dialkylated lariat ether. Fluorescence intensity was recorded each second using a Horiba Fluoromax 4 spectrometer (excitation wavelength=640 nm; emission wavelength=670 nm). The solution inside the cuvette was vigorously mixed throughout the experiment by using a magnetic stirrer and the temperature was kept constant at 25° C. To normalize the fluorescence, experiments in the same conditions but without adding bacteria were performed, normalization was done by dividing the fluorescence by the final fluorescence level of these experiments.

Cell hyperpolarization (more negatively charged inside the cell) results in an uptake of the dye, while cell membrane depolarization (more positively charged inside the cell) results in a release of the dye. The accumulation of the dye in the interior of the cell can be detected by a decrease in fluorescence due to self-quenching, which enables the dye to be utilized as an indirect reporter of changes in cell membrane voltage. As the resting membrane voltage in B. subtilis is approximately −120 mV, DiSC₃(5) quickly accumulates inside intact bacteria (timepoint t2 in FIGS. 23-31). The addition of up to 60 mM KCl (timepoint t3 in FIGS. 23-31) does not cause substantial membrane depolarization, as determined by the limited increase in fluorescence, mainly because the endogenous K⁺ transporters and channel activities are inhibited at 25° C. However, upon addition of the dialkylated lariat ether (timepoint t4 in FIGS. 23-31), ions move down the electrochemical gradient and give rise to membrane depolarization, as evidenced by an increase in fluorescence.

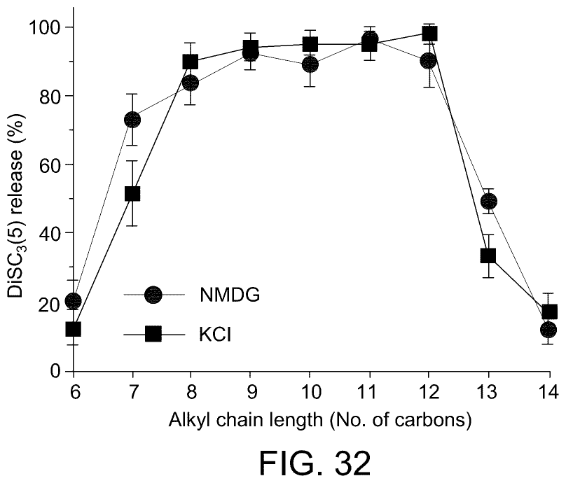

FIG. 32 shows relative DiSC₃(5) release after 10 min of treatment with 2 μM of various dialkylated lariat ethers in the presence of 60 mM KCl or 60 mM NMDG-Cl. Values are the average±S.E.M. of values measured at the end of at least three experiments, similar to those described in FIGS. 23-31. The effects of the alkyl chain length on the relative DiSC₃(5) release after 10 minutes following addition of the dialkylated lariat ether (shown in FIG. 32) were qualitatively similar to those reported previously.

The dialkylated lariat ethers with the highest toxicities elicited faster DiSC₃(5) efflux in the presence of K⁺, suggesting they are more efficient at transporting cations (FIGS. 23-32). As the induction of membrane depolarization is consistent with an ionophoric mechanism, the next step was to determine the cation selectivity exhibited by this class of the compounds.

Binding to ionophores requires at least a partial substitution of water molecules in the hydration sphere by ions to achieve an ionophore-like transport mechanism. Thus, some degree of ion selectivity is expected, as observed in the case of valinomycin, a natural carrier-type ionophore that is extremely selective for K⁺ over Na⁺ and does not transport N-methyl-d-glucamine (NMDG⁺).

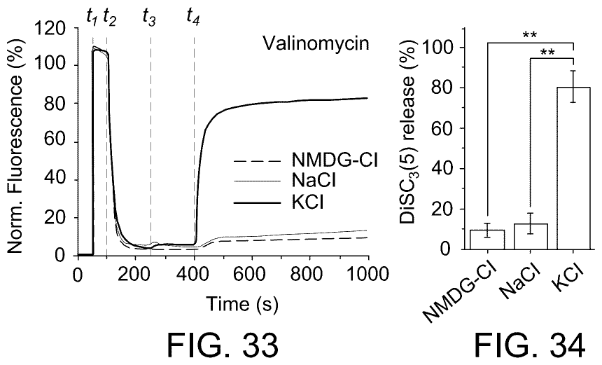

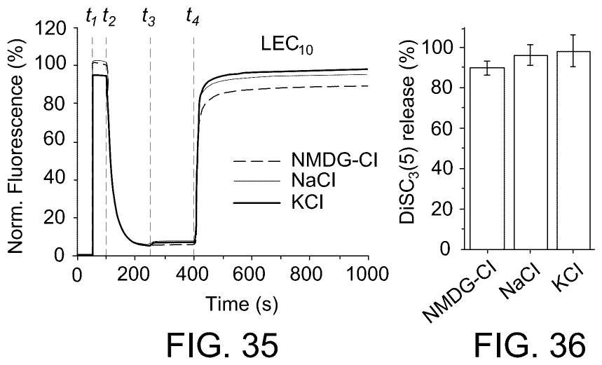

Referring to FIGS. 33-36, a comparison between the selectivity of valinomycin and LEC₁₀ is shown. FIG. 33 shows a time course of normalized changes in DiSC₃(5) fluorescence due to the activity of 2 μM valinomycin. The experiment was conducted by successive additions of dye (t1), B. subtilis cells (t2), concentrated salts up to 60 mM KCl, NaCl or 60 mM NMDG-Cl (t3), and valinomycin up to 2 μM (t4). FIG. 34 shows fluorescence values at the end of the experiment of FIG. 33 (average±S.E.M). FIG. 35 shows a time course of normalized changes in DiSC₃(5) fluorescence due to the activity of 2 μM LEC₁₀; the experiment is identical to that in FIG. 33, except LEC₁₀ was added in t4. FIG. 36 shows fluorescence at the end of the experiment as shown in FIG. 35 (average±S.E.M). A minimum of three experiments for each condition were averaged in FIGS. 34 and 36.

As shown in FIGS. 33 and 34, when depolarization assays were performed in the presence of different cations, valinomycin promoted DiSC₃(5) release only when K⁺ was added to the external solution, but not when Na⁺ or NMDG⁺ was added. Conversely, as shown in FIGS. 35 and 36, when LEC₁₀ was tested under the same conditions, no significant differences in relative DiSC₃(5) release were observed (see FIGS. 35 and 36). Furthermore, the control experiments show that even in the absence of the alkali cation, the relative DiSC₃(5) release rates were similar to those observed in the presence of K⁺ (FIGS. 23-32).

There are two possible explanations for this unexpected result. Either the dialkylated lariat ethers behave as non-selective ionophores that are capable of transporting large cations, such as NMDG⁺, or their primary effect is to disrupt membrane integrity, i.e., the DiSC₃(5) efflux is due to the lysis of the cells, rather than ion transport across the membrane.

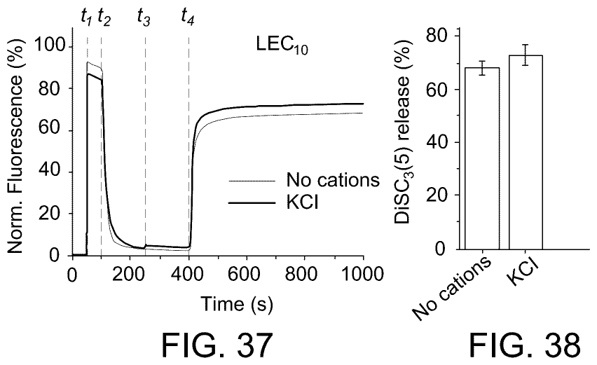

Referring to FIGS. 37 and 38, to determine whether transport of NMDG⁺ can account for the observed efflux of DiSC₃(5) from cells, LEC₁₀ activity was tested in a cation-free dextrose solution. FIG. 37 shows a time course of the DiSC₃(5) fluorescence due to the activity of LEC₁₀. This experiment is identical to that shown in FIG. 27, with the exception that it was performed in a cation-free dextrose solution (200 mM dextrose, 10 mM Tris, pH adjusted to 7.4 with methanesulfonic acid). The events in FIG. 37 are: t1, addition of dye; t2, addition of B. subtilis; t3, addition of 2 M KCl until a final concentration of 60 mM was reached or the same volume of additional dextrose solution; t4, addition of LEC₁₀ up to a final concentration of 2 μM. Traces were normalized as described above. FIG. 38 shows relative DiSC₃(5) release after 10 min of treatment with 2 μM LEC₁₀ in the presence or the absence of 60 mM KCl. The values are averages±S.E.M. of at least three experiments, similar to those described in FIG. 37.

In this experiment, the only cation in the external solution is added at timepoint t3 (see FIG. 37). Addition of 2 μM LEC₁₀ produced a large DiSC₃(5) release in the presence of KCl, but interestingly, this same effect was also observed in the absence of any external cation (FIG. 37). The independence of the DiSC₃(5) efflux from the identity or the presence of the external cations is not compatible with an ionophore-like mechanism.

Planar Lipid Bilayer Experiments

Previous studies indicated that lariat ethers reported to display the ability to collapse membrane potential in depolarization assays also exhibited ion channel-like activity in bilayers. This suggested that similar depolarization assays may be employed to serve as a convenient surrogate for measuring electrical activity. The most potent compound, LEC₁₀, was tested for ion channel activity in planar lipid bilayers.

Experiments in planar lipid bilayers were performed via a Nanion Orbit-mini planar bilayer system. The planar membranes were formed by painting a 25 mg/mL solution of lipids in n-nonane over the four 50 μm apertures on the chip. The lipids used were asolectin from soybean (Sigma-Aldrich) or a 3:1 w/w mix of 1-palmitoyl-2-oleoyl-sn-glycero-3-phosphoethanolamine (POPE) and 1-palmitoyl-2-oleoyl-sn-glycero-3-phospho-(1′-rac-glycerol) (POPG) (Avanti Polar lipids). The solutions tested were KCl solution (150 mM KCl, 10 mM HEPES, pH adjusted to 7.4 with KOH), NaCl solution (150 mM NaCl, 10 mM HEPES, pH adjusted to 7.4 with NaOH), and a concentrated KCl solution (500 mM KCl, 10 mM HEPES, pH adjusted to 7.4 with KOH). When the appropriate membrane formed, the voltage was clamped at 100 mV or −100 mV, and a stock solution of gramicidin D (Sigma-Aldrich) in DMSO or LEC₁₀ in TFE were added up to the desired concentration. The tested concentration of gramicidin D was 1 nM and 2, 10, 100 and 200 μM were tested for LEC₁₀. Two different temperatures were tested for each condition: 25 and 37° C. The current was recorded over 10 min for the gramicidin test and up to 1 h for the LEC₁₀ test. The data was analyzed with Clampfit 10.7 software (Axon Instruments).

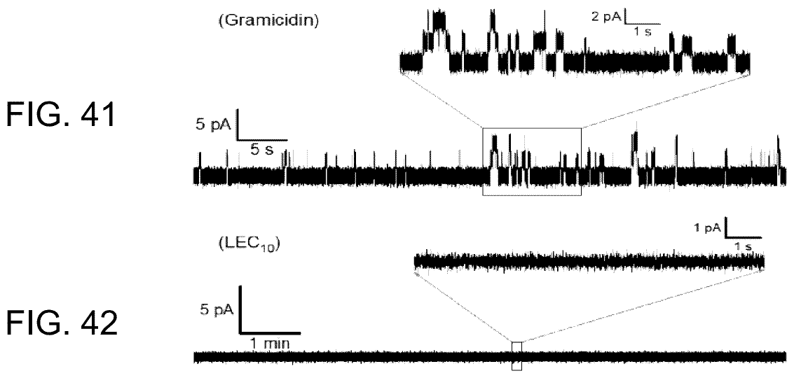

FIGS. 41 and 42 show the lipid bilayer recordings. Current through the asolectin planar lipid bilayer clamped at 100 mV was recorded in the presence of 2 μM gramicidin (FIG. 41) and 2 μM LEC₁₀ (FIG. 42). In both cases, the solution was 150 mM KCl, 10 mM HEPES, pH 7.4. The unitary channels of the ionophore gramicidin recorded in the presence of a 150 mM KCl solution (FIG. 41) served as a control. However, no ion channel activity was detected in a similar experiment performed using the 2 μM LEC₁₀ (FIG. 42), even after one hour of recording. Variations in the LEC₁₀ concentration (10, 100, and 200 μM), as well as the KCl concentration (up to 500 mM), including replacing KCl with NaCl, resulted in no indication of ion channel activity. This lack of unitary channel formation, or even a carrier-like increase of the conductance, precludes an ionophore-like mechanism of ion transport.

Lactate Dehydrogenase Activity Determination

In view of the findings that the class of dialkylated lariat ether derivatives described herein do not act as typical ionophores, the possibility that the activity of these compounds results from disruption of membrane integrity was considered. A well-established assay was utilized to measure cell lysis by monitoring the release of the enzyme lactate dehydrogenase (LDH), which is ubiquitous in the cytoplasm of all cell types. The tetrameric active form of LDH catalyzes the final step of the glycolysis in B. subtilis and has a molecular weight of approximately 146 kDa in, with a nearly globular shape of an approximated radius of 80 Å (PDB ID: 3PQD). The release of proteins of this size demonstrates the test compounds are likely to cause cell lysis but the possibility that they form large pores cannot be ruled out. LDH couples two redox reactions, the first involving the interconversion of pyruvate (oxidized) and L-lactate (reduced) and the second, the interconversion of NAD⁺ (oxidized) and NADH (reduced). The NADH oxidation can be coupled to the diaphorase-catalyzed oxidation of resazurin to resofurin, which is highly fluorescent. When the concentrations of L-lactate, NAD⁺, diaphorase, and resazurin are saturated, the rate of increase in the resofurin fluorescence is limited only by the amount of available LDH in the medium.

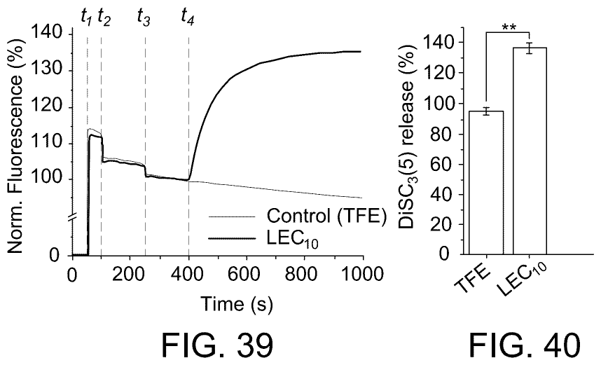

To prepare the experiment, a 2-mL liquid culture of B. subtilis cells was grown at 37° C. in media until OD₆₀₀=0.600, and then collected by centrifugation at 2000 rpm during 3 min. Bacteria were washed once in lactate/NAD⁺ solution (95 mM lithium lactate, 10 mM NAD⁺, 10 mM HEPES, pH adjusted to 7.4 with LiOH). The centrifugation step was repeated and the bacteria were resuspended until OD₆₀₀=1.0 and then diluted 100× in lactate/NAD⁺ solution. The working fluorophore solution was a 1 mM solution of resazurin sodium salt (Alfa Cesar) in water. The experiment was started with 3 mL of lactate/NAD⁺ solution in a quartz cuvette, then 3.1 μL of resazurin solution were added (timepoint t1, 50 sec; see FIG. 39) for a 1 μM final concentration, followed by the addition of 100 μL of the diluted B. subtilis suspension (timepoint t2, 100 sec; FIG. 39). After 150 sec (timepoint t3, 250 sec; FIG. 39) 10 μL of the diaphorase (Worthintong) solution was added. The final step involved the addition of 0.62 μL of a 10 mM stock solution of LEC₁₀ in TFE (timepoint t4, 400 sec; FIG. 39), for a final LEC₁₀ concentration of 2 μM. Fluorescence intensity was recorded each second using a Horiba Fluoromax 4 spectrometer (excitation wavelength=550 nm; emission wavelength=583 nm). The solution inside the cuvette was vigorously mixed throughout the experiment by using a magnetic stirrer and the temperature was kept constant at 37° C. As a control, one experiment was performed under the same conditions, except the same amount of TFE was added without the dialkylated lariat ether, up to a final concentration of 0.02%. To normalize, the fluorescence was divided by the fluorescence level just before timepoint t4.

Referring to FIGS. 39 and 40, LDH release from B. subtilis in response to LEC₁₀ is shown. FIG. 39 shows the normalized time course of the resofurin fluorescence. The experiment was conducted by successive additions of resazurin (t1); B. subtilis cells (t2), diaphorase (t3), and up to 2 μM LEC₁₀ or the same volume of TFE (control) (t4). FIG. 40 shows normalized resofurin fluorescence at the end of the experiment as shown in FIG. 39 (average±S.E.M).

The experiments show that the addition of LEC₁₀ to a mixture containing B. subtilis cells and the other components necessary for the LDH assay causes a rapid rise in the fluorescence (FIGS. 39 and 40), indicating loss of membrane integrity. In contrast, the addition of the same amount of the trifluoroethanol (TFE) solvent used to dissolve LEC₁₀ did not produce any increase in the fluorescence.

B. Self-Assembling, Monosubstituted Benzo(Crown-Ether) Compounds that Exhibit Ion Channel Activity in Biological Membranes

The preceding examples demonstrate that various dialkylated lariat ethers, which were previously assumed to form membrane channels, are actually membrane-lytic and do not exhibit channel activity in lipid bilayers. The examples below demonstrate that self-assembling monoacylated and monoalkylated benzo(crown-ether) compounds, which lack the ability to engage in H-bonding, display robust channel activity.

Synthesis and Preparation of Monoacylated and Monoalkylated Benzo(Crown-Ether) Compounds

The following procedure is representative of the general procedure for monosubstituted benzo(crown-ether) formation. In the representative procedure, m is an integer from 1 to 3, and n is an integer from 0 to 19, such as an integer from 2 to 9, or n is an integer selected from the group consisting of 2, 4, 6, 8, and 9.

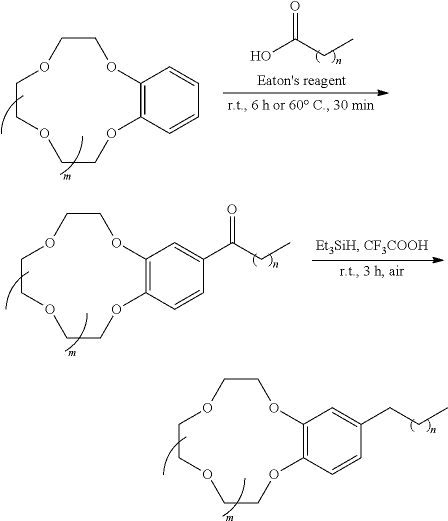

Commercially available benzo(12-crown-4), (15-crown-5), and (18-crown-6) ether compounds (TCI) are exposed to carboxylic acids of varying tail lengths in the presence of an acid catalyst (e.g., Eaton's reagent) to furnish the corresponding acylated benzo(crown-ether) derivatives. The acylated derivatives are reduced in suitable conditions with a reducing agent, such as by adding a hydrosilane (e.g., triethyl silane) to a solution of the acylated benzo(crown-ether) in acid (e.g., trifluoracetic acid) to yield the alkylated benzo(crown-ether) compounds. The structures are confirmed by Nuclear Magnetic Resonance (NMR) and mass spectrometry (MS). In the example monosubstituted benzo(crown-ether) compounds described further below, each acylated benzo(crown-ether) compound and alkylated benzo(crown-ether) is identified with the code “X-Y-Z”, where X is the number of heteroatoms in the ring (e.g., 4, 5, or 6), Y is the letter O if the carbonyl is present and A if it is not, and Z is the length of the acyl or alkyl chain (e.g., 4-11 carbons).

I. Synthesis and Purification of Monoacylated Benzo(Crown-ether)

The following procedure is representative of the general procedure for monoacylated benzo(crown-ether) formation. In the representative procedure, R may be a C₁₋₂₀alkyl chain, straight chain or branched, optionally unsaturated, where R is not substituted by any hydrogen bond donors. In the example compounds disclosed herein, R is a straight chain, saturated C₃₋₁₀alkyl chain. Further details of the procedure are provided in example Procedure A and Procedure B below.

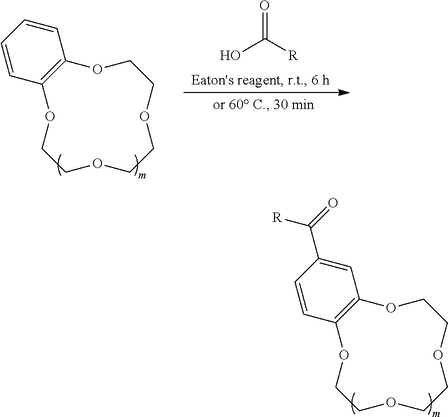

Procedure A: A mixture of (1 equiv.) benzo(crown-ether) and (1.4 equiv.) of appropriate carboxylic acid in Eaton's reagent (10 ml, 0.1 M) was stirred at room temperature for 6 h. The reaction mixture eventually turned dark red. The mixture was then poured into 100 ml of cold H₂O and stirred for 10-30 min, and then neutralized via dropwise addition of 100 mL NaHCO₃. The solution was extracted with CH₂Cl₂ (3×100 mL) and the combined organics were washed with DI-H₂O (1×100 mL) and 1M NaOH (1×100 mL), dried over Na₂SO₄ and concentrated under reduced pressure. The crude product was purified by recrystallization with hexane (generally up to 96% yield). If the solid did not form upon water quench, chloroform was added and the aqueous mixture transferred to a separatory funnel. The aqueous layer was washed with chloroform (3×100 mL), and the organic layers combined, washed with brine, dried with magnesium sulfate and concentrated in vacuo. The crude material was then purified via silica gel chromatography, eluting with hexanes/ethyl acetate (100:0 to 0:100 gradient).

Procedure B: A mixture of benzo(crown-ether) (1 equiv.) and the appropriate carboxylic acid (1 equiv.) were combined in Eaton's reagent (10 mL) and heated to between 50 and 60° C. and stirred vigorously until a bright cherry red color was observed throughout. Approximately 20 min after the development of the bright cherry red color, the reaction was quenched by pouring into ice water (˜50 g). (Note that although heating up to an hour results in no loss in yield, the longer the system is heated past the 20-30 min window, the greater the byproduct formation, resulting in erosion of the yield). In most cases, the solid precipitate was isolated via filtration and recrystallized from ethanol to give the product as a white solid. If solid did not form upon the water quench, chloroform was added and the aqueous mixture was transferred to a separatory funnel. The aqueous layer was washed with 3× portions of chloroform, the organic layers combined, washed with brine, dried with magnesium sulfate and concentrated in vacuo. The crude material was then purified via silica gel chromatography eluting with hexanes/ethyl acetate (100:0 to 0:100 gradient).

In both Procedure A and Procedure B, the freshness of Eaton's Reagent is paramount to high yields. As the reagent decays, the yield steadily decreases until the reaction gives no product formation. This is despite the fact that the reagent may still be viable in other transformations.

The general procedure described in Procedure A is suitable for most compounds reported herein, however, heating the reaction mixture (e.g., to a temperature between 40-100° C., between 50-90° C., between 50-60° C., or between 60-90° C.) allows for completion of the reaction in less than or equal to 1 hour and facilitates the formation of fewer byproducts.

The development of a uniform cherry red color indicates that the reaction is unlikely to undergo further conversion to product, which in some cases occurs prior to the 5 hour mark with minor loss in yield. Shorter reaction time without full red color results in a loss of yield. A purple color has also been observed, with no difference in yield or product purity.

The physical state for many of these compounds is concentration and scale dependent. For example, synthesis of 1-(2,3,5,6,8,9,11,12,14,15-decahydrobenzo[b][1,4,7,10,13,16]hexaoxacyclooctadecin-18-yl)undecan-1-one (6-O-11) at 0.25 mmol scale or less results in an oily substance upon quenching with water (see below) requiring aqueous workup. However, at 0.5 mmol and larger scales, 6-O-11 precipitates reliably as a white solid. In general, it has been observed that larger scales tend to give crystalline material.

The benzo(crown-ether) is prone to aggregate formation in NMR solvent, which leads to additional peaks and high variation in shifts. Suitably, spectra are obtained in a more dilute form to ensure sharper peaks and more reproducible chemical shifts.

Example monoacylated benzo(crown-ether) compounds will now be described. All compounds were synthesized according to the above-described procedures and had spectral data consistent with reported compounds. ¹H NMR and ¹³C NMR spectra were obtained using Bruker Avance-500 spectrometers. Chemical shifts are reported relative to the tetramethylsilane peak (δ 0.00 ppm). Accurate mass measurements were acquired at the University of Wisconsin, Madison, using a Micromass LCT (electrospray ionization, time-of-flight analyzer or electron impact methods).

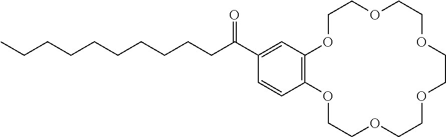 [SMILES: CCCCCCCCCCC(=O)c1ccc2c(c1)OCCOCCOCCOCCOCCO2]

1-(2,3,5,6,8,9,11,12,14,15-decahydrobenzo[b][1,4,7,10,13,16]hexaoxacyclooctadecin-18-yl)undecan-1-one (6-O-11). Following general Procedure B. The reaction was performed on 1.6 mmol scale to provide the product as an off-white solid (18%, 0.256 g) after column chromatography. ¹H NMR (400 MHz, Chloroform-d) δ 7.54 (dd, J=8.4, 2.0 Hz, 1H), 7.50 (d, J=2.0 Hz, 1H), 6.85 (d, J=8.3 Hz, 1H), 4.20 (dd, J=5.6, 3.3 Hz, 4H), 3.95-3.90 (m, 4H), 3.80-3.73 (m, 4H), 3.73-3.63 (m, 8H), 2.88 (t, J=7.5 Hz, 2H), 1.76-1.61 (m, 2H), 1.47-1.17 (m, 14H), 0.91-0.79 (m, 3H). ¹³C NMR (101 MHz, Chloroform-d) δ 199.4, 153.2, 148.7, 130.6, 123.0, 112.8, 111.9, 71.0, 71.0, 70.9, 70.8, 70.8, 70.7, 69.6, 69.4, 69.2, 69.0, 38.3, 32.0, 29.6, 29.6, 29.5, 29.4, 29.4, 24.8, 22.8, 14.2. HRMS (ESI) m/z calculated for C₂₇H₄₄O₇ [M+Na]⁺ 503.2979, found 503.2975.

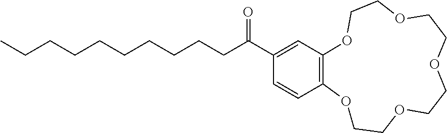 [SMILES: CCCCCCCCCCC(=O)c1ccc2c(c1)OCCOCCOCCOCCO2]

1-(2,3,5,6,8,9,11,12-octahydrobenzo[b][1,4,7,10,13]pentaoxacyclopentadecin-15-yl)undecan-1-one (5-O-11). Following general Procedure B. The reaction was performed on 0.5 mmol scale to provide the product as a yellow solid (69%, 0.150 g) after recrystallization from hexanes. ¹H NMR (400 MHz, Chloroform-d) δ 7.54 (dd, J=8.4, 2.0 Hz, 1H), 7.49 (d, J=2.1 Hz, 1H), 6.83 (d, J=8.4 Hz, 1H), 4.22-4.11 (m, 4H), 3.92-3.88 (m, 4H), 3.74 (d, J=2.5 Hz, 8H), 2.87 (t, J=7.5 Hz, 2H), 1.72-1.65 (m, 2H), 1.42-1.17 (m, 14H), 0.87-0.81 (m, 3H). ¹³C NMR (101 MHz, Chloroform-d) δ 199.4, 153.3, 148.9, 130.6, 123.1, 112.8, 111.8, 71.2 (2×CH₂), 70.5, 70.4, 69.5, 69.3, 69.0, 68.7, 38.3, 32.0, 29.7, 29.6, 29.6, 29.5, 29.4, 24.8, 22.8, 14.2. HRMS (ESI) m/z calculated for C₂₅H₄₀O₆ [M+Na]⁺ 459.2717, found 459.2711.

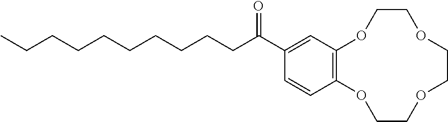 [SMILES: CCCCCCCCCCC(=O)c1ccc2c(c1)OCCOCCOCCO2]

1-(2,3,5,6,8,9-hexahydrobenzo[b][1,4,7,10]tetraoxacyclododecin-12-yl)undecan-1-one (4-O-11). Following general Procedure A. The reaction was conducted on 0.5 mmol scale. After quenching with water, white solids precipitated but dissolved upon standing leaving a clear colorless solution with oil like clumps distributed within. An equal volume of DCM was added to the water mixture and transferred to a separatory funnel. The organic layer was separated and washed with 1M NaOH (1×20 mL), DI water (1×20 mL) then dried over sodium sulfate, filtered, and concentrated in vacuo to give a yellow oil. The oil was dissolved in DCM and applied to silica gel column (Hexanes/EtOAc) and isolated as a pale yellow amorphous solid (16%, 0.063 g). ¹H NMR (400 MHz, Chloroform-d) δ 7.58 (s, 1H), 6.95-6.88 (m, 2H), 4.21-4.15 (m, 4H), 4.15-4.10 (m, 1H), 3.86-3.81 (m, 2H), 3.81-3.75 (m, 3H), 3.73 (d, J=7.1 Hz, 5H), 2.84 (t, J=7.4 Hz, 2H), 1.66 (t, J=7.3 Hz, 2H), 1.34-1.15 (m, 14H). ¹³C NMR (101 MHz, Chloroform-d) δ199.0, 155.1, 150.1, 131.5, 124.1, 118.5, 115.7, 72.6, 71.3, 70.9, 70.7, 69.7, 69.6, 38.3, 31.9, 29.6, 29.5, 29.5, 29.4, 29.3, 24.6, 22.6, 14.1. HRMS (ESI) m/z calculated for C₂₃H₃₆O₅ [M+H]⁺ 393.2636, found 393.2632.

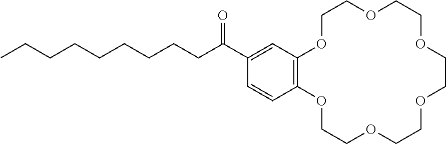 [SMILES: CCCCCCCCCC(=O)c1ccc2c(c1)OCCOCCOCCOCCOCCO2]

1-(2,3,5,6,8,9,11,12,14,15-decahydrobenzo[b][1,4,7,10,13,16]hexaoxacyclooctadecin-18-yl)decan-1-one (6-O-10). Following the general Procedure A. The reaction was conducted on 0.5 mmol scale to provide the product as an off-white solid (50 mg, 25% yield). ¹H NMR (600 MHz, Chloroform-d) δ 7.56 (dd, J=8.4, 2.0 Hz, 1H), 7.52 (d, J=2.0 Hz, 1H), 6.87 (d, J=8.4 Hz, 1H), 4.21 (t, 4H), 3.94 (m, 4H), 3.78 (m, 4H), 3.72 (t, J=6.0, 4.0, 1.8 Hz, 4H), 3.69 (m, 4H), 2.89 (t, 2H), 1.70 (sextet, J=7.4 Hz, 2H), 1.29-1.24 (m, 12H), 0.88 (t, J=7.0 Hz, 3H). ¹³C NMR (151 MHz, Chloroform-d) δ 199.33, 152.79, 148.67, 130.49, 122.93, 112.80, 111.90, 71.00, 70.96, 70.85, 70.76, 70.73, 70.67, 69.51, 69.38, 69.13, 68.94, 38.22, 31.89, 29.51, 29.49, 29.45, 29.30, 24.76, 22.68, 14.12. HRMS (ESI) m/z calculated for C₂₆H₄₂O₇ [M+Na]⁺489.2820, found 489.2823.

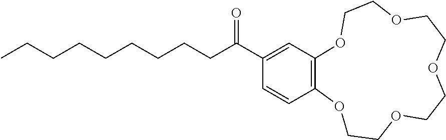 [SMILES: CCCCCCCCCC(=O)c1ccc2c(c1)OCCOCCOCCOCCO2]

1-(2,3,5,6,8,9,11,12-octahydrobenzo[b][1,4,7,10,13]pentaoxacyclopentadecin-15-yl)decan-1-one (5-O-10). Following the general Procedure A, the reaction was conducted on 0.5 mmol scale to provide product as a white solid (107 mg, 54% yield). ¹H NMR (600 MHz, Chloroform-d) δ 7.56 (dd, J=8.4, 2.0 Hz, 1H), 7.51 (d, J=2.0 Hz, 1H), 6.85 (d, J=8.3 Hz, 1H), 4.19 (t, J=5.6, 3.1 Hz, 4H), 3.92 (t, J=8.8, 3.2 Hz, 4H), 3.77 (m, 8H), 2.90 (t, J=7.4 Hz, 2H), 1.70 (m, J=7.4 Hz, 2H), 1.40-1.20 (m, 12H), 0.88 (t, J=6.7 Hz, 3H). ¹³C NMR (151 MHz, Chloroform-d) δ 199.13, 155.09, 150.12, 131.59, 124.16, 118.54, 115.74, 72.69, 72.68, 71.41. 71.40, 70.94, 70.74, 69.79, 69.68, 38.33, 31.89, 29.51, 29.49, 29.42, 29.30, 24.62, 22.68, 14.12. HRMS (ESI) m/z calculated for C₂₄H₃₈O₆ [M+Na]⁺445.2559, found 445.2561.

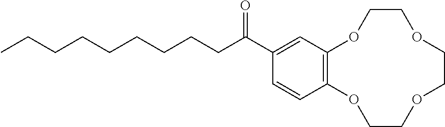 [SMILES: CCCCCCCCCC(=O)c1ccc2c(c1)OCCOCCOCCO2]

1-(2,3,5,6,8,9-hexahydrobenzo[b][1,4,7,10]tetraoxacyclododecin-12-yl)decan-1-one (4-O-10). Following general Procedure A, the reaction was conducted on 0.5 mmol scale to provide product as an off-white solid (111 mg, 56% yield). ¹H NMR (600 MHz, Chloroform-d) δ 7.65-7.62 (m, 2H), 6.97 (d, J=7.8 Hz, 1H), 4.23 (m, 4H), 3.90-3.82 (m, 4H), 3.78 (m, 4H), 2.91-2.88 (t, 2H), 1.70 (m, J=7.2 Hz, 2H), 1.46-1.17 (m, 12H), 0.89-0.86 (t, 3H). ¹³C NMR (151 MHz, Chloroform-d) δ 199.13, 155.09, 150.12, 131.59, 124.16, 118.54, 115.74, 72.69, 71.41, 70.94, 70.74, 69.79, 69.68, 38.33, 31.89, 29.51, 29.49, 29.42, 29.30, 24.62, 22.68, 14.12. HRMS (ESI) m/z calculated for C₂₂H₃₄O₅ [M+Na]⁺ 401.2113, found 401.2295.

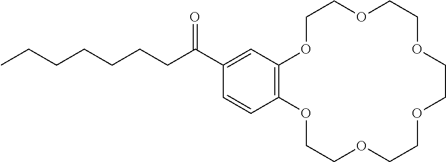 [SMILES: CCCCCCCC(=O)c1ccc2c(c1)OCCOCCOCCOCCOCCO2]

1-(2,3,5,6,8,9,11,12,14,15-decahydrobenzo[b][1,4,7,10,13,16]hexaoxacyclooctadecin-18-yl)octan-1-one (6-O-8). Following general Procedure A, the reaction was conducted on 0.5 mmol scale. The product was isolated as an off-white solid (60 mg, 27%). ¹H NMR (400 MHz, Chloroform-d) δ 7.56 (dd, J=8.4, 2.1 Hz, 1H), 7.52 (d, J=2.0 Hz, 1H), 6.86 (d, J=8.4 Hz, 1H), 4.25-4.19 (m, 4H), 3.96-3.92 (m, 4H), 3.80-3.75 (m, 4H), 3.73-3.71 (m, 4H), 3.69 (s, 4H), 2.90 (t, J=7.5 Hz, 2H), 1.72 (p, J=7.3 Hz, 2H), 1.46-1.18 (m, 11H), 0.93-0.88 (m, 3H). ¹³C NMR (101 MHz, Chloroform-d) δ 199.4, 153.1, 148.7, 130.5, 123.0, 112.6, 111.8, 71.0, 70.9, 70.8, 70.8, 70.7, 70.7, 69.5, 69.4, 69.0, 68.9, 38.3, 31.8, 29.6, 29.2, 24.8, 22.7, 14.2. HRMS (ESI) m/z calculated for C₂₄H₃₈O₇ [M+NH₄]⁺ 456.2956, found 456.2950.

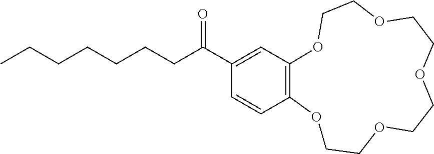 [SMILES: CCCCCCCC(=O)c1ccc2c(c1)OCCOCCOCCOCCO2]

1-(2,3,5,6,8,9,11,12-octahydrobenzo[b][1,4,7,10,13]pentaoxacyclopentadecin-15-yl)octan-1-one (5-O-8). Following general Procedure A, the reaction was conducted on 0.5 mmol scale. The product was isolated as an off-white solid (79 mg, 40%). ¹H NMR (400 MHz, Chloroform-d) δ 7.54 (dd, J=8.4, 2.1 Hz, 1H), 7.49 (d, J=2.1 Hz, 1H), 6.83 (d, J=8.4 Hz, 1H), 4.22-4.12 (m, 4H), 3.96-3.85 (m, 4H), 3.74 (bd, J=2.6 Hz, 8H), 2.87 (t, J=7.4 Hz, 2H), 1.75-1.63 (m, 2H), 1.39-1.19 (m, 8H), 0.91-0.80 (m, 3H). ¹³C NMR (101 MHz, Chloroform-d) δ 199.4, 153.4, 148.9, 130.6, 123.1, 112.8, 111.8, 71.3 (2×CH₂), 70.5, 70.4, 69.5, 69.4, 69.0, 68.7, 38.3, 31.8, 29.5, 29.2, 24.8, 22.7, 14.2. HRMS (ESI) m/z calculated for C₂₂H₃₄O₆ [M+Na]⁺ 417.2248, found 417.2240.

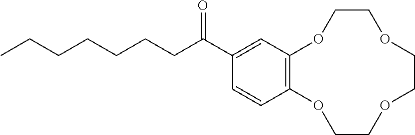 [SMILES: CCCCCCCC(=O)c1ccc2c(c1)OCCOCCOCCO2]

1-(2,3,5,6,8,9-hexahydrobenzo[b][1,4,7,10]tetraoxacyclododecin-12-yl)octan-1-one (4-O-8). Following general Procedure A, the reaction was conducted on 0.5 mmol scale. The product was isolated as an off-white solid (74 mg, 42%). ¹H NMR (400 MHz, Chloroform-d) δ 7.66-7.59 (m, 2H), 6.97 (d, J=8.9 Hz, 1H), 4.26-4.18 (m, 4H), 3.93-3.86 (m, 2H), 3.86-3.80 (m, 2H), 3.78 (s, 4H), 2.89 (t, J=7.4 Hz, 2H), 1.71 (p, J=7.4 Hz, 2H), 1.43-1.20 (m, 8H), 0.92-0.81 (m, 3H). 13C NMR (101 MHz, Chloroform-d) δ 199.1, 155.1, 150.1, 131.6, 124.2, 118.5, 115.7, 72.7, 71.4, 70.9, 70.7, 69.8, 69.7, 38.3, 31.7, 29.4, 29.2, 24.6, 22.6, 14.1. HRMS (ESI) m/z calculated for C₂₀H₃₀O₅ [M+H]⁺ 351.2166, found 351.2161.

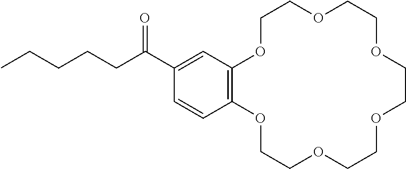 [SMILES: CCCCCC(=O)c1ccc2c(c1)OCCOCCOCCOCCOCCO2]

1-(2,3,5,6,8,9,11,12,14,15-decahydrobenzo[b][1,4,7,10,13,16]hexaoxacyclooctadecin-18-yl)hexan-1-one (6-O-6). Following the general Procedure A, the reaction was conducted on 0.5 mmol scale to provide product as a white solid (90 mg, 45% yield). ¹H NMR (500 MHz, Chloroform-d) δ 7.56 (dd, J=8.3, 2.0 Hz, 1H), 7.52 (d, J=2.0 Hz, 1H), 6.87 (d, J=8.3 Hz, 1H), 4.22 (m, 4H), 3.98-3.91 (m, 4H), 3.80-3.77 (m, 4H), 3.76-3.70 (m, 4H), 3.69 (m, 4H), 2.89 (t, J=7.4 Hz, 2H), 1.75-1.69 (m, 2H), 1.37-1.34 (m, 4H), 0.91 (t, 3H). ¹³C NMR (126 MHz, Chloroform-d) δ 199.29, 153.17, 148.71, 130.54, 122.93, 112.93, 112.01, 71.01, 70.97, 70.86, 70.77 (2×CH₂), 70.70, 69.54, 69.41, 69.19, 68.98, 38.16, 31.62, 24.44, 22.54, 13.97. HRMS (ESI) m/z calculated for C₂₂H₃₄O₇ [M+Na]⁺433.2123, found 433.2197.

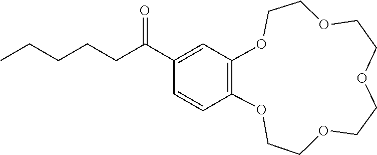 [SMILES: CCCCCC(=O)c1ccc2c(c1)OCCOCCOCCOCCO2]

1-(2,3,5,6,8,9,11,12-octahydrobenzo[b][1,4,7,10,13]pentaoxacyclopentadecin-15-yl)hexan-1-one (5-O-6). Following the general Procedure A, the reaction was conducted on 0.5 mmol scale to provide product as a white solid (46 mg, 23% yield). ¹H NMR (500 MHz, Chloroform-d) δ 7.56 (dd, J=8.3, 2.0 Hz, 1H), 7.51 (d, J=2.0 Hz, 1H), 6.85 (d, J=8.4 Hz, 1H), 4.23-4.16 (m, 4H), 3.95-3.89 (m, 4H), 3.85-3.68 (m, 8H), 2.90 (t, 2H), 1.72 (t, J=7.4, 4.3, 2.0 Hz, 2H), 1.38-1.33 (m, 4H), 0.91 (t, 3H). ¹³C NMR (126 MHz, Chloroform-d) δ 199.29, 153.31, 148.85, 130.56, 122.99, 112.85, 111.83, 71.24, 71.23, 70.47, 70.37, 69.45, 69.33, 69.01, 68.71, 38.16, 31.62, 24.44, 22.55, 13.98. HRMS (ESI) m/z calculated for C₂₀H₃₀O₆ [M+Na]⁺389.1935, found 389.1935.

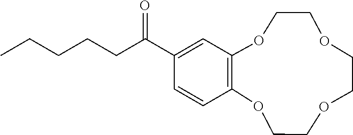 [SMILES: CCCCCC(=O)c1ccc2c(c1)OCCOCCOCCO2]

1-(2,3,5,6,8,9-hexahydrobenzo[b][1,4,7,10]tetraoxacyclododecin-12-yl)hexan-1-one (4-O-6). Following the general Procedure A, the reaction was conducted on 0.5 mmol scale to provide product as a white solid (94 mg, 47% yield). ¹H NMR (500 MHz, Chloroform-d) δ 7.63 (m, J=7.9 Hz, 2H), 6.97 (dd, 1H), 4.26-4.21 (m, 4H), 3.92-3.82 (m, 4H), 3.80-3.76 (m, 4H), 2.89 (t, J=7.4 Hz, 2H), 1.75-1.69 (m, 2H), 1.38-1.33 (m, 4H), 0.92 (t, 3H). ¹³C NMR (126 MHz, Chloroform-d) δ 199.14, 155.10, 150.17, 131.64, 124.15, 118.55, 115.80, 72.72, 71.47, 71.02, 70.82, 69.84, 69.75, 38.29, 31.60, 24.31, 22.55, 13.97. HRMS (ESI) m/z calculated for C₁₈H₂₆O₅ [M+Na]⁺345.1660, found 345.1666.

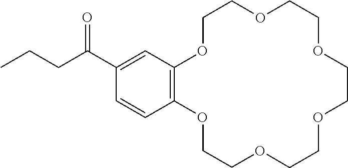 [SMILES: CCCC(=O)c1ccc2c(c1)OCCOCCOCCOCCOCCO2]

1-(2,3,5,6,8,9,11,12,14,15-decahydrobenzo[b][1,4,7,10,13,16]hexaoxacyclooctadecin-18-yl)butan-1-one (6-O-4). Following general Procedure A, the reaction was conducted on 1.0 mmol scale. The product was isolated as an off-white solid after column chromatography (124 mg, 32%). ¹H NMR (400 MHz, Chloroform-d) δ 7.66-7.45 (m, 2H), 6.87 (d, J=8.5 Hz, 1H), 4.21 (t, J=4.3 Hz, 4H), 4.00-3.85 (m, 4H), 3.85-3.61 (m, 12H), 2.89 (t, J=7.3 Hz, 2H), 1.75 (bq, J=7.4 Hz, 2H), 0.99 (t, J=7.5 Hz, 3H). ¹³C NMR (101 MHz, Chloroform-d) δ 199.0, 153.1, 148.6, 130.4, 122.9, 112.6, 111.8, 70.9, 70.8, 70.7, 70.7, 70.6, 70.6, 69.4, 69.3, 69.0, 68.8, 40.0, 18.0, 13.9. HRMS (ESI) m/z calculated for C₂₀H₃₀O₇ [M+NH₄]⁺ 400.2328, found 400.2328.

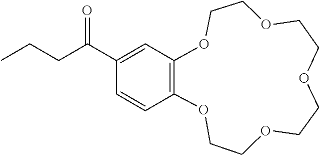 [SMILES: CCCC(=O)c1ccc2c(c1)OCCOCCOCCOCCO2]

1-(2,3,5,6,8,9,11,12-octahydrobenzo[b][1,4,7,10,13]pentaoxacyclopentadecin-15-yl)butan-1-one (5-O-4). Following general Procedure A, the reaction was conducted on 1.0 mmol scale. The product was isolated as an off-white waxy solid after column chromatography (166 mg, 49.1%). ¹H NMR (400 MHz, Chloroform-d) δ 7.50 (d, J=8.3 Hz, 1H), 7.45 (s, 1H), 6.79 (d, J=8.3 Hz, 1H), 4.12 (bs, 4H), 3.83 (bs, 4H), 3.70 (s, 8H), 2.82 (t, J=7.3 Hz, 2H), 1.69 (h, J=7.4 Hz, 2H), 0.93 (t, J=7.5 Hz, 3H). ¹³C NMR (101 MHz, Chloroform-d) δ 199.0, 153.2, 148.7, 130.4, 122.9, 112.7, 111.7, 71.1 (2×CH₂), 70.3, 70.2, 69.3, 69.2, 68.9, 68.6, 40.0, 18.0, 13.9. HRMS (ESI) m/z calculated for C₁₈H₂₆O₆ [M+Na]⁺361.1622, found 361.1616.

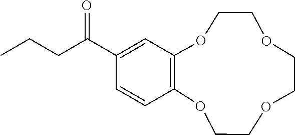 [SMILES: CCCC(=O)c1ccc2c(c1)OCCOCCOCCO2]

1-(2,3,5,6,8,9,11,12-octahydrobenzo[b][1,4,7,10,13]pentaoxacyclopentadecin-15-yl)butan-1-one (4-O-4). Following general Procedure A, the reaction was conducted on 1.0 mmol scale. The product was isolated as an off-white solid after column chromatography (191 mg, 64.9%). ¹H NMR (400 MHz, Chloroform-d) δ 7.66-7.59 (m, 2H), 6.97 (d, J=9.0 Hz, 1H), 4.26-4.18 (m, 4H), 3.91-3.84 (m, 2H), 3.85-3.78 (m, 2H), 3.76 (s, 4H), 2.88 (t, J=7.3 Hz, 2H), 1.74 (h, J=7.4 Hz, 2H), 0.99 (t, J=7.4 Hz, 3H). ¹³C NMR (101 MHz, Chloroform-d) δ 198.6, 154.8, 149.8, 131.2, 123.9, 118.2, 115.5, 72.4, 71.0, 70.5, 70.3, 69.4, 69.3, 39.9, 17.7, 13.6. HRMS (ESI) m/z calculated for C₁₆H₂₂O₅ [M+H]⁺ 295.1540, found 295.1536.

II. Synthesis and Purification of Monoalkylated Benzo(Crown-Ether)

The following procedure is representative of the general procedure for monoalkylated benzo(crown-ether) formation. In the representative procedure, R may be a C₁₋₂₀alkyl chain, straight chain or branched, optionally unsaturated, where R is not substituted by any hydrogen bond donors. In the example compounds disclosed herein, R is a straight chain, saturated C₃₋₁₀alkyl chain. Further details of the procedure are provided in example Procedure C below.

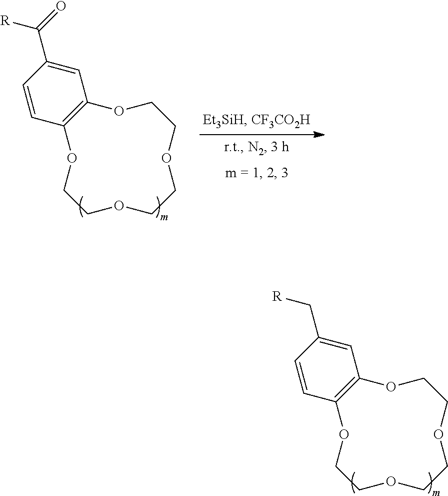

Procedure C: Triethylsilane (10 equiv.) was added to a solution of monosubstituted benzo(crown-ethers) (1 equiv.) in trifluoracetic acid (1 equiv.). The solution was allowed to stir at room temperature for 3 h under an inert atmosphere. The reaction mixture was then diluted with chloroform (75 mL) and saturated bicarbonate was added slowly until the effervescence ceased. The organic layer was separated and washed with DI water (2×15 mL), dried over Na₂SO₄, and concentrated in vacuo. The solid residue was recrystallized from ethanol to give the title compound as a white solid. For non-solid compounds, trituration with toluene and concentration on high vac for up to 4 days was required in some cases to remove the excess triethyl silane.

Example monoalkylated benzo(crown-ether) compounds will now be described. All compounds were synthesized according to the above-described procedures and had spectral data consistent with reported compounds. ¹H NMR and ¹³C NMR spectra were obtained using Bruker Avance-500 spectrometers. Chemical shifts are reported relative to the tetramethylsilane peak (δ0.00 ppm). Accurate mass measurements were acquired at the University of Wisconsin, Madison, using a Micromass LCT (electrospray ionization, time-of-flight analyzer or electron impact methods).

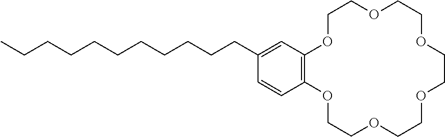 [SMILES: CCCCCCCCCCCc1ccc2c(c1)OCCOCCOCCOCCOCCO2]

18-undecyl-2,3,5,6,8,9,11,12,14,15-decahydrobenzo[b][1,4,7,10,13,16]hexaoxacyclooctade-cine (6-A-11). Following the general Procedure C, the reaction was conducted on 0.06 mmol scale. The product was isolated as a yellow oil (7.4 mg, 24%). ¹H NMR (500 MHz, Chloroform-d) δ 6.83-6.77 (m, 1H), 6.71 (bs, 2H), 4.44-4.32 (m, 3H), 4.19-4.09 (m, 3H), 3.91-3.62 (m, 10H), 3.07 (d, J=9.3 Hz, 4H), 2.52 (t, J=7.8 Hz, 2H), 1.65-1.50 (m, 2H), 1.33-1.24 (m, 16H), 0.88 (t, J=6.9 Hz, 3H). ¹³C NMR (126 MHz, CDCl₃) δ 148.4, 146.7, 136.6, 121.2, 114.7, 114.4, 70.7, 69.9, 69.9, 69.5, 69.3, 69.1, 69.0, 68.8, 68.8, 68.6, 37.8, 37.7, 35.5, 31.9, 31.7, 29.7, 29.6, 29.5, 29.3, 22.7, 14.1. HRMS (ESI) m/z calculated for C₂₇H₄₆O₆ [M+Na]⁺ 489.3192, found 489.3189.

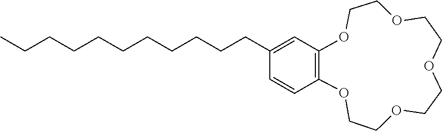 [SMILES: CCCCCCCCCCCc1ccc2c(c1)OCCOCCOCCOCCO2]

15-undecyl-2,3,5,6,8,9,11,12-octahydrobenzo[b][1,4,7,10,13]pentaoxacyclopentadecine (5-A-11). Following the general Procedure C, the reaction was conducted on 0.14 mmol scale. The product was isolated as an off-white waxy solid (28.6 mg, 53%). ¹H NMR (500 MHz, Chloroform-d) δ 6.72 (d, J=8.6 Hz, 1H), 6.66-6.57 (m, 2H), 4.10-4.03 (m, 4H), 3.91-3.81 (m, 4H), 3.73-3.62 (m, 8H), 2.50-2.34 (m, 2H), 1.54-1.44 (m, 2H), 1.19 (s, 16H), 0.83-0.79 (m, 3H). ¹³C NMR (126 MHz, Chloroform-d) δ 148.9, 147.0, 136.4, 120.9, 114.6, 114.3, 71.0 (2×CH₂), 70.6, 70.6, 69.7, 69.7, 69.3, 69.0, 35.5, 31.9, 31.6, 29.7, 29.6, 29.6, 29.5, 29.4, 29.3, 22.7, 14.1. HRMS (ESI) m/z calculated for C₂₅H₄₂O₅ [M+Na]⁺445.2925, found 445.2924.

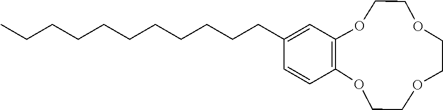 [SMILES: CCCCCCCCCCCc1ccc2c(c1)OCCOCCOCCO2]

12-undecyl-2,3,5,6,8,9-hexahydrobenzo[b][1,4,7,10]tetraoxacyclododecine (4-A-11). Following the general Procedure C, the reaction was conducted on 0.16 mmol scale. The product was isolated as an off-white waxy solid (54.0 mg, 100%). ¹H NMR (400 MHz, Chloroform-d) δ 6.89 (d, J=8.0 Hz, 1H), 6.81-6.71 (m, 2H), 4.24-4.11 (bs, 4H), 3.85 (bs, 4H), 3.8 (bs, 4H) 2.52 (t, J=7.7 Hz, 2H), 1.63-1.48 (m, 2H), 1.27 (bs, 16H), 0.88 (t, J=6.7 Hz, 3H). ¹³C NMR (101 MHz, Chloroform-d) δ 150.4, 148.4, 138.1, 122.5, 118.4, 118.1, 72.2, 71.5, 71.1, 71.0, 70.0 (2×CH₂), 35.6, 32.1, 31.7, 29.8, 29.8, 29.7, 29.6, 29.5, 29.4, 22.8, 14.3. HRMS (ESI) m/z calculated for C₂₃H₃₈O₄ [M+Na]⁺401.2662, found 401.2660.

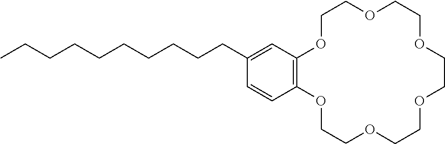 [SMILES: CCCCCCCCCCc1ccc2c(c1)OCCOCCOCCOCCOCCO2]

18-decyl-2,3,5,6,8,9,11,12,14,15-decahydrobenzo[b][1,4,7,10,13,16]hexaoxacyclooctadecine (6-A-10). Following the general Procedure C, the reaction was conducted on 1 mmol scale to provide product as a light-yellow oil (27 mg, 53% yield). ¹H NMR (500 MHz, Chloroform-d) δ 6.79 (d, J=8.0 Hz, 1H), 6.73-6.67 (m, 2H), 4.18-4.11 (m, 4H), 3.88 (m, 4H), 3.80-3.75 (m, 4H), 3.75-3.66 (m, 4H), 3.70-3.67 (m, 4H), 2.51 (t, 2H), 1.56 (m, 2H), 1.34-1.20 (m, 14H), 0.87 (t, J=7.1 Hz, 3H). ¹³C NMR (126 MHz, Chloroform-d) δ 148.96, 147.06, 136.51, 121.01, 115.05, 114.78, 70.90, 70.88 (2×CH₂), 70.85, 70.82, 69.88, 69.85, 69.56, 69.39, 69.31, 35.50, 31.92, 31.63, 29.64, 29.38, 29.31, 29.27, 22.70, 22.68, 14.13. HRMS (ESI) m/z calculated for C₂₆H₄₄O₆ [M+Na]⁺475.4325, found 475.3025.

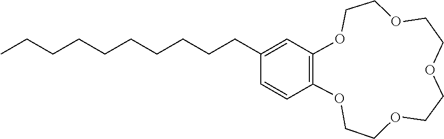 [SMILES: CCCCCCCCCCc1ccc2c(c1)OCCOCCOCCOCCO2]

15-decyl-2,3,5,6,8,9,11,12-octahydrobenzo[b][1,4,7,10,13]pentaoxacyclopentadecine (5-A-10). Following the general Procedure C, the reaction was conducted on 0.2 mmol scale to provide product as a light-yellow oil (30 mg, 43% yield). ¹H NMR (500 MHz, Chloroform-d) δ 6.79 (d, J=8.1 Hz, 1H), 6.69 (m, J=7.6 Hz, 2H), 4.16-4.09 (m, 4H), 3.94-3.87 (m, 4H), 3.80-3.73 (m, 8H), 2.51 (t, 2H), 1.59-1.53 (m, 2H), 1.34-1.27 (m, 14H), 0.88 (t, 3H). ¹³C NMR (126 MHz, Chloroform-d) δ 149.03, 147.12, 136.42, 120.91, 114.69, 114.45, 71.10, 71.09, 70.68, 70.64, 69.81, 69.77, 69.44, 69.13, 31.86, 31.64, 29.71, 29.40, 29.34, 29.29, 29.07, 22.67, 14.12, 14.10. HRMS (ESI) m/z calculated for C₂₄H₄₀O₅ [M+Na]⁺431.2758, found 431.2768.

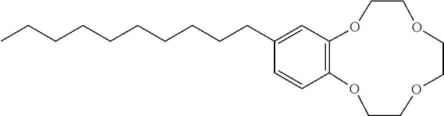 [SMILES: CCCCCCCCCCc1ccc2c(c1)OCCOCCOCCO2]

12-decyl-2,3,5,6,8,9-hexahydrobenzo[b][1,4,7,10]tetraoxacyclododecine (4-A-10). Following the general Procedure C, the reaction was conducted on 0.2 mmol scale to provide product as a white solid (56 mg, 80% yield). ¹H NMR (500 MHz, Chloroform-d) δ 6.89 (d, J=8.1 Hz, 1H), 6.79 (d, J=2.1 Hz, 1H), 6.75 (dd, J=8.1, 2.1 Hz, 1H), 4.20-4.13 (m, 4H), 3.89-3.82 (m, 4H), 3.81 (m, 4H), 2.55-2.49 (t, 2H), 1.56 (m, J=7.4 Hz, 2H), 1.32-1.28 (m, 14H), 0.88 (t, J=6.9 Hz, 3H). ¹³C NMR (126 MHz, Chloroform-d) δ 150.31, 148.26, 137.98, 122.38, 118.30, 117.99, 72.11, 71.42, 71.09, 70.94, 69.91, 69.90, 35.47, 31.92, 31.55, 29.64, 29.62, 29.52, 29.35 (2×CH₂), 29.31, 22.70, 14.13. HRMS (ESI) m/z calculated for C₂₂H₃₆O₄ [M+Na]⁺387.2553, found 387.2506.

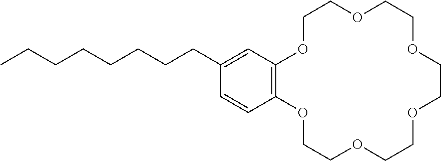 [SMILES: CCCCCCCCc1ccc2c(c1)OCCOCCOCCOCCOCCO2]

18-octyl-2,3,5,6,8,9,11,12,14,15-decahydrobenzo[b][1,4,7,10,13,16]hexaoxacyclooctadecine (6-A-8). Following general Procedure C, the reaction was conducted on 0.10 mmol scale. The product was isolated as a waxy solid after column chromatography (30.5 mg, 67%). ¹H NMR (400 MHz, Chloroform-d) δ 6.84-6.74 (m, 1H), 6.70 (d, J=7.1 Hz, 2H), 4.20-4.08 (m, 4H), 4.00-3.88 (m, 4H), 3.78-3.75 (m, 4H), 3.72-3.70 (m, 4H), 3.68 (bs, 4H), 2.51 (t, J=7.7 Hz, 2H), 1.56 (bs, 2H), 1.37-1.17 (m, 10H), 0.88 (t, J=6.8 Hz, 3H). ¹³C NMR (101 MHz, Chloroform-d) δ 148.6, 146.7, 136.5, 121.0, 114.5, 114.2, 70.7 (3×CH₂), 70.6 (3×CH₂), 69.6 (2×CH₂), 69.0, 68.8, 35.6, 32.0, 31.7, 29.8, 29.6, 29.4, 22.8, 14.2. HRMS (ESI) m/z calculated for C₂₄H₄₀O₆ [M+Na]⁺447.2717, found 447.2714.

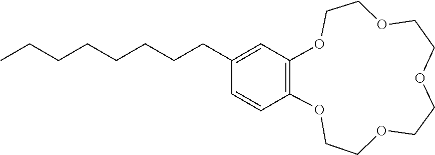 [SMILES: CCCCCCCCc1ccc2c(c1)OCCOCCOCCOCCO2]

15-octyl-2,3,5,6,8,9,11,12-octahydrobenzo[b][1,4,7,10,13]pentaoxacyclopentadecine (5-A-8). Following general Procedure C, the reaction was conducted on 0.16 mmol scale. The product was isolated as a waxy solid after column chromatography (28.6 mg, 53%). ¹H NMR (400 MHz, Chloroform-d) δ 6.79 (d, J=8.6 Hz, 1H), 6.71 (d, J=6.2 Hz, 2H), 4.20-4.08 (m, 4H), 3.93 (q, J=4.8 Hz, 4H), 3.82-3.68 (m, 8H), 2.56-2.47 (m, 2H), 1.57-1.45 (m, 2H), 1.26 (m, 10H), 0.88 (t, J=6.7 Hz, 3H). ¹³C NMR (101 MHz, Chloroform-d) δ 148.4, 146.5, 136.6, 121.1, 114.2, 113.9, 70.5 (2×CH₂), 70.3, 70.2, 69.2, 69.2, 68.8, 68.5, 35.6, 34.2, 32.0, 31.7, 29.6, 29.4, 22.8, 14.2. HRMS (ESI) m/z calculated for C₂₂H₃₆O₅ [M+Na]⁺403.2455, found 403.2446.

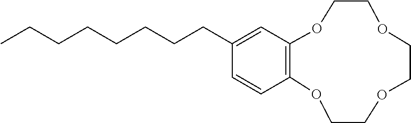 [SMILES: CCCCCCCCc1ccc2c(c1)OCCOCCOCCO2]

12-octyl-2,3,5,6,8,9-hexahydrobenzo[b][1,4,7,10]tetraoxacyclododecine (4-A-8). Following general Procedure C, the reaction was conducted on 0.16 mmol scale. The product was isolated as a waxy solid after column chromatography (54 mg, 100%). ¹H NMR (400 MHz, Chloroform-d) δ 6.89 (d, J=8.0 Hz, 1H), 6.82-6.72 (m, 2H), 4.17 (t, J=6.1 Hz, 4H), 3.91-3.75 (m, 8H), 2.52 (t, J=7.7 Hz, 2H), 1.63-1.52 (m, 2H), 1.27 (d, J=9.3 Hz, 10H), 0.88 (t, J=6.6 Hz, 3H). ¹³C NMR (101 MHz, Chloroform-d) δ 150.4, 148.4, 138.0, 122.4, 118.4, 118.1, 72.2, 71.5, 71.2, 71.0, 70.0 (2×CH₂), 35.6, 32.0, 31.7, 29.6, 29.4, 29.4, 22.8, 14.2. HRMS (ESI) m/z calculated for C₂₀H₃₂O₄ [M+Na]⁺359.2193, found 359.2189.

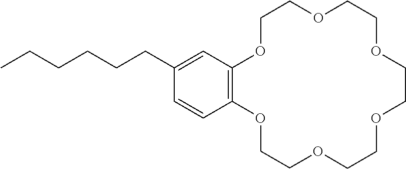 [SMILES: CCCCCCc1ccc2c(c1)OCCOCCOCCOCCOCCO2]

18-hexyl-2,3,5,6,8,9,11,12,14,15-decahydrobenzo[b][1,4,7,10,13,16]hexaoxacyclooctadecine (6-A-6). Following the general Procedure C, the reaction was conducted on 0.5 mmol scale to provide product as a white solid (14 mg, 70% yield). ¹H NMR (500 MHz, Chloroform-d) δ 6.80 (d, 1H), 6.76-6.67 (m, 2H), 4.19-4.09 (m, 4H), 3.95-3.87 (m, 4H), 3.81-3.75 (m, 4H), 3.72 (m, 4H), 3.68 (m, 4H), 2.55-2.48 (t, 2H), 1.60-1.52 (m, 2H), 1.30-1.23 (m, 6H), 0.88 (t, J=4.8 Hz, 3H). ¹³C NMR (126 MHz, Chloroform-d) δ 148.92, 147.03, 136.48, 120.99, 114.98, 114.70, 70.88, 70.87, 70.86 (2×CH₂) 70.83, 70.80, 69.86, 69.83, 69.50, 69.26, 35.49, 31.74, 31.57, 28.95, 22.62, 14.10. HRMS (ESI) m/z calculated for C₂₂H₃₆O₆ [M+Na]⁺419.2452, found 416.2404.

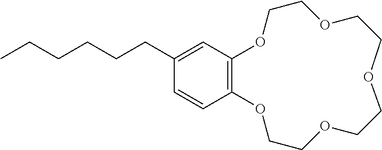 [SMILES: CCCCCCc1ccc2c(c1)OCCOCCOCCOCCO2]

15-hexyl-2,3,5,6,8,9,11,12-octahydrobenzo[b][1,4,7,10,13]pentaoxacyclopentadecine (5-A-6). Following the general Procedure C, the reaction was conducted on 0.4 mmol scale to yield product as a white solid (80 mg, 62% yield). ¹H NMR (500 MHz, Chloroform-d) δ 6.81-6.76 (m, 1H), 6.69 (d, J=7.2 Hz, 2H), 4.17-4.07 (m, 4H), 3.94-3.87 (m, 4H), 3.80-3.72 (m, 8H), 2.57-2.48 (t, 2H), 1.60-1.52 (m, 2H), 1.33-1.28 (m, 6H), 0.92-0.85 (t, 3H). ¹³C NMR (126 MHz, Chloroform-d) δ 149.02, 147.11, 136.42, 120.92, 114.69, 114.45, 71.09, 70.72, 70.66, 70.62, 69.79, 69.75, 69.42, 69.12, 35.52, 31.75, 31.59, 28.95, 22.63, 14.11. HRMS (ESI) m/z calculated for C₂₀H₃₂O₅ [M+Na]⁺375.2147, found 375.2142.

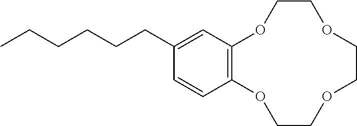 [SMILES: CCCCCCc1ccc2c(c1)OCCOCCOCCO2]

12-hexyl-2,3,5,6,8,9-hexahydrobenzo[b][1,4,7,10]tetraoxacyclododecine (4-A-6). Following the general Procedure C, the reaction was conducted on 0.2 mmol scale to yield product as a light-yellow solid (114 mg, 57% yield). ¹H NMR (500 MHz, Chloroform-d) δ 6.88 (d, J=8.1 Hz, 1H), 6.83-6.71 (m, 2H), 4.21-4.12 (m, 4H), 3.90-3.81 (m, 4H), 3.80 (m, 4H), 2.57-2.49 (t, 2H), 1.61-1.52 (m, 2H), 1.37-1.24 (m, 6H), 0.88 (t, 3H). ¹³C NMR (126 MHz, Chloroform-d) δ 150.43, 148.39, 137.87, 122.29, 118.30, 118.00, 72.23, 71.53, 71.27, 71.12, 70.03, 70.02, 35.47, 31.73, 31.50, 28.96, 22.62, 14.10. HRMS (ESI) m/z calculated for C₁₈H₂₈O₄ [M+Na]⁺331.1842, found 331.1880.

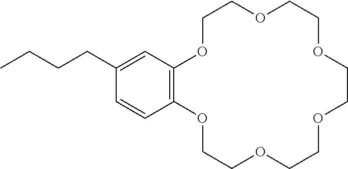 [SMILES: CCCCc1ccc2c(c1)OCCOCCOCCOCCOCCO2]

Mono-benzo 18-crown-6-ether (6-A-4). Following the general Procedure C, the reaction was conducted on 0.16 mmol scale. The product was isolated as an off-white solid after column chromatography and two days under high vacuum to remove triethyl silane impurities (31.1 mg, 52%). ¹H NMR (400 MHz, Chloroform-d) δ 6.79-6.61 (m, 3H), 4.19-4.03 (m, 4H), 3.83 (s, 4H), 3.70-3.52 (m, 11H), 2.49 (t, J=7.7 Hz, 2H), 1.59-1.47 (m, 2H), 1.36-1.17 (m, 6H). ¹³C NMR (101 MHz, Chloroform-d) δ 146.7, 144.9, 136.8, 121.2, 112.5, 112.0, 69.1 (2×CH₂), 68.8 (2×CH₂), 68.7 (2×CH₂), 68.5 (2×CH₂), 66.3, 66.1, 35.2, 33.7, 22.2, 13.8. HRMS (ESI) m/z calculated for C₂₀H₃₂O₆ [M+Na]⁺391.2091, found 391.2088.

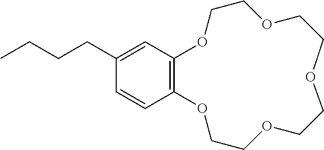 [SMILES: CCCCc1ccc2c(c1)OCCOCCOCCOCCO2]

15-butyl-2,3,5,6,8,9,11,12-octahydrobenzo[b][1,4,7,10,13]pentaoxacyclopentadecine (5-A-4). Following the general Procedure C, the reaction was conducted on 0.25 mmol scale. The product was isolated as an off-white solid after column chromatography (70.1 mg, 88%). ¹H NMR (400 MHz, Chloroform-d) δ 6.73 (d, J=8.1 Hz, 1H), 6.70-6.58 (m, 2H), 4.17-4.02 (m, 4H), 3.88 (d, J=6.2 Hz, 3H), 3.65 (d, J=29.1 Hz, 8H), 2.45 (t, J=7.7 Hz, 2H), 1.55-1.34 (m, 2H), 1.30-1.16 (m, 2H), 0.83 (t, J=7.4 Hz, 3H). ¹³C NMR (101 MHz, Chloroform-d) δ 146.8, 144.9, 137.1, 121.5, 113.2, 112.9, 69.0 (2×CH₂), 68.7 (2×CH₂), 67.8, 67.8, 67.3, 67.0, 35.2, 33.7, 22.3, 13.9. HRMS (ESI) m/z calculated for C₁₈H₂₈O₅ [M+Na]⁺347.1829, found 347.1827.

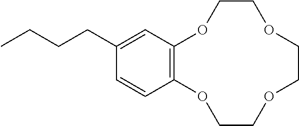 [SMILES: CCCCc1ccc2c(c1)OCCOCCOCCO2]

12-butyl-2,3,5,6,8,9-hexahydrobenzo[b][1,4,7,10]tetraoxacyclododecine (4-A-4). Following the general Procedure C, the reaction was conducted on 1.0 mmol scale. The product was isolated as an off-white solid after column chromatography (79.9 mg, 82%). ¹H NMR (500 MHz, Chloroform-d) δ 6.91 (d, J=8.2 Hz, 1H), 6.84-6.75 (m, 2H), 4.27-4.16 (m, 4H), 3.78-3.65 (m, 8H), 2.52 (t, J=7.8 Hz, 2H), 1.54 (p, J=7.5 Hz, 2H), 1.37-1.30 (m, 2H), 0.95-0.87 (m, 3H). ¹³C NMR (126 MHz, Chloroform-d) δ 148.8, 146.8, 138.8, 123.2, 118.2, 117.9, 70.4, 69.8, 67.6 (2×CH₂), 67.3, 67.3, 35.1, 33.6, 22.4, 13.9. HRMS (ESI) m/z calculated for C₁₆H₂₄O₄ [M+Na]⁺ 303.1567, found 303.1563.

### Experimental Results

To investigate whether the hydrogen bonding networks that were previously implicated in the formation of supramolecular columnar structures are essential for channel activity, the following experiments were conducted. The series of MAcBCE and MAkBCE compounds disclosed herein, which lack the ability to engage in H-bonding, were synthesized according to the procedures described above. In contrast to other previous versions of benzo(crown-ether), these compounds lack the ureido group previously implicated as essential to H-bonding and columnar assembly but nonetheless were found to display robust channel activity. Surprisingly, single-channel recordings showed that the activity of the compounds is ion-dependent, yet ion selectivity measurements reveal that the channels are essentially non-selective with respect to Na⁺ and K⁺. These findings reveal an unexpected variety of ions on the channel activity of benzo(crown-ether) compounds and may account for previously reported putative ion selectivities in macroscopic studies.

Toxicity on Bacteria

XL1 blue strain Escherichia coli cells transformed with pET28a plasmid containing a kanamycin resistance gene, were grown at 37° C. in 2 mL of LB medium (Miller's LB broth, Research Products International) supplemented with 50 μg/mL of kanamycin, until the optical density (OD₆₀₀) reached 0.6. The starting density of bacteria in the remainder of the experiments was a 1/100 dilution of this bacterial density, which was split into 4 mL cultures. A series of 400, 100, 10 and 5 mM stock solutions of each monoacylated benzo(crown-ether) (MAcBCE) and monoalkylated benzo(crown-ether) (MAkBCE) compounds in trifluoroethanol (TFE) were diluted by addition to the 4 mL cultures to reach final concentrations of 400, 200, 100, 50, 20, 15, 10, 5, 4, 3, 2 and 1 μM. The final TFE concentration was never higher than 0.1% v/v. Negative controls in the presence of TFE 0.1% v/v or in the absence of any treatment for each batch gave similar bacterial growth. The cultures were incubated at 37° C. with agitation for 12 h. Bacillus subtilis (168 WT) toxicity tests were carried out using an identical protocol, except they were transformed with the shuttle plasmid pRB374 in the presence of 5 μg/mL of kanamycin. For both bacterial species, the minimum inhibitory concentrations (MIC) were determined as the lowest compound concentration that inhibited growth after 12 h, as judged by visual turbidity. Each compound was assayed a minimum of three times at each tested concentration.

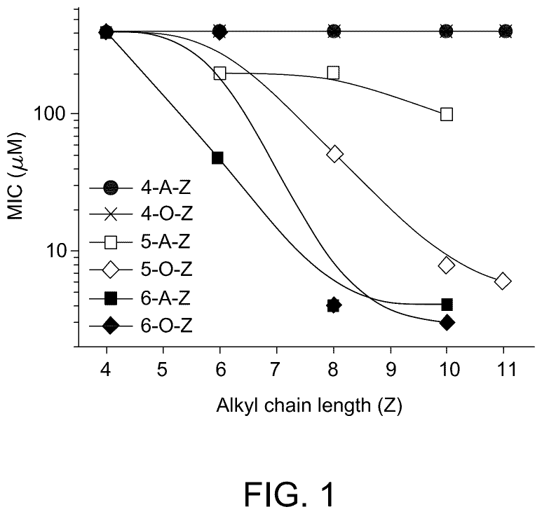

The cytotoxicity of MABCE compounds was determined by measuring the MIC in liquid cultures of either the gram-negative bacteria Escherichia coli or the gram-positive bacteria Bacillus subtilis. FIG. 1 shows the MIC for benzo(12-crown-4) (4-A-Z and 4-O-Z), benzo(15-crown-5) (5-A-Z and 5-O-Z), and benzo(18-crown-6) (6-A-Z and 6-O-Z) ether compounds on B. subtilis. As some curves overlap, portions of them are hidden. Values of MIC of 400 μM indicate that the value was either 400 μM, or it was higher and then was not determined. Each compound was assayed a minimum of three times at each different concentration. MICs for 5-A-4, 5-A-11, 6-A-11 and 6-O-11 could not be determined because the compounds were not soluble in TFE. While E. coli was not susceptible to any of the tested compounds at concentrations as high as 400 μM (data not shown), B. subtilis was susceptible to MABCE compounds bearing 15-crown-5 and 18-crown-6 scaffolds, but not to compounds containing a 12-crown-4 scaffold, as shown in FIG. 1. For the 15-crown-5 and 18-crown-6 compounds, the MIC decreased with the length of the chain, establishing chain length as another critical determinant of toxicity in addition to the size of the crown ether.

The results shown in FIG. 1 demonstrate that MAcBCE and MAkBCE compounds are toxic to the gram-positive B. subtilis bacteria. The outer membrane of gram-negative bacteria is known to represent a formidable barrier to antibiotics. It is not surprising that the MAcBCE and MAkBCE compounds are toxic only to the gram-positive bacteria. Without being limited to any particular theory, it is thought that the outer membrane poses a barrier for the MAcBCE and MAkBCE compounds, which are likely to accumulate and thus display limited impact on the permeability of inner membrane.

Lactate Dehydrogenase Activity Determination

A 2 mL liquid culture of transformed B. subtilis cells was grown at 37° C. in LB media supplemented with 5 μM kanamycin, until OD₆₀₀=0.6. Cells were collected by centrifugation at 2000 rpm over 3 min. The bacterial pellet was washed twice in lactate/NAD⁺ solution (95 mM potassium lactate, 10 mM nicotinamide adenine dinucleotide (NAD⁺), 10 mM HEPES, pH adjusted to 7.4 with KOH). After the last centrifugation step, the bacteria were resuspended until OD₆₀₀=1.0 in the lactate/NAD⁺ solution, then diluted 100× in the same solution. The working fluorophore solution was a 1 mM of resazurin sodium salt (Alfa Cesar) in water. The experiment was started with 3 mL of lactate/NAD⁺ solution in a quartz cuvette, then 3.1 μL of resazurin solution was added (timepoint t₁, 50 sec) for a 1 μM final concentration, followed by the addition of 100 μL of the diluted B. subtilis suspension (timepoint t₂, 100 sec). After 150 sec (timepoint t₃, 250 sec), 10 μL of diaphorase (Worthinton Biochemical Corporation) 10 mg/mL solution was added. The final step involved the addition of 0.62 μL of a 10 mM stock solution of dialkylated diaza lariat ether (LEC₁₀) in TFE (timepoint t₄, 400 sec), for a final LEC₁₀ concentration of 2 μM; or 3.1 μL of a 2 mM stock solution of valinomycin in DMSO, for a final valinomycin concentration of 2 μM; or 3.1 μL of a 20 mg/mL stock solution of lysozyme in lactate/NAD⁺ solution, for a final lysozyme concentration of 20 μg/mL; or 3.1 μL of 10 mM stock solution of each MAcBCE or MAkBCE compounds in TFE, for final concentrations of 10 μM. Fluorescence intensity was recorded each second using a Horiba Fluoromax 4 spectrometer (λ_{excitation}=550 nm; λₑₘᵢₛₛᵢₒₙ=583 nm). The solution inside the cuvette was vigorously mixed throughout the experiment by using a magnetic stirrer and the temperature was kept constant at 37° C. As a negative control, one experiment was performed by adding 3.1 μL of pure TFE, up to a final concentration of 0.1% v/v. To normalize, the fluorescence intensity was divided by the fluorescence level just before timepoint t₄.

Some bioactive lariat ether compounds can be actually membrane lytic agents instead ionophores. The addition of these compounds to cells at concentrations corresponding to their normal working range effectively released the cytoplasmic enzyme lactate dehydrogenase (LDH; a 146 kDa globular protein folds with 160 Å diameter (PDB ID: 3PQD)), indicating that the membrane integrity was compromised. To test if the effects of MAcBCE and MAkBCE compounds were due to lytic activity, a resofurin-based fluorescent assay was performed to detect the LDH release.

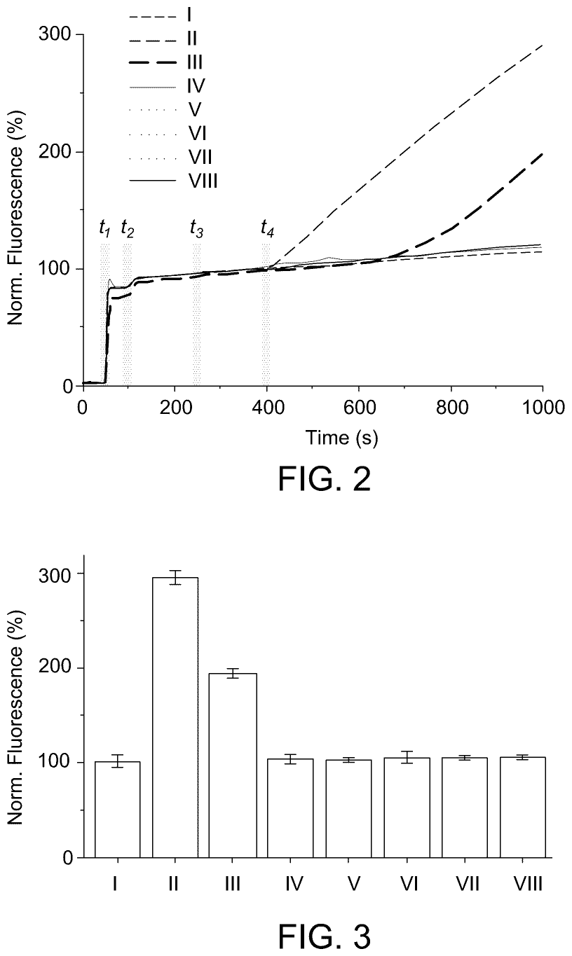

FIG. 2 shows the normalized time course of the resofurin fluorescence in response to the treatment with I: 0.1% v/v TFE, II: 2 μM LEC₁₀, III: 20 μg/mL lysozyme, IV: 2 μM valinomycin, V: 10 μM 5-A-10, VI: 10 μM 5-O-10, VII: 10 μM 6-A-10, and VIII: 10 μM 6-O-10. The experiments were performed in a lactate/NAD⁺ solution (95 mM potassium lactate, 10 mM NAD⁺, 10 mM HEPES, pH adjusted to 7.4 with KOH). The events are: t₁, addition of resazurin; t₂, addition of B. subtilis cells; t₃, addition of diaphorase; t₄, addition of the tested compound (colored curves) or the same volume of TFE (control, grey curve).

FIG. 3 shows the normalized resofurin fluorescence at the end of the experiments (mean±S.E.M). Experiments were repeated at least three times per condition. To normalize, the fluorescence intensity was divided by the fluorescence level just before timepoint t₄.

As shown in FIGS. 2 and 3, while the addition of LEC₁₀ (a crown ether based lytic agent) or lysozyme clearly led to an increase in the resofurin fluorescence levels, the K⁺ carrier valinomycin and the bioactive MAcBCE and MAkBCE compounds had no effect. MAcBCE and MAkBCE compounds self-assemble into ion channels but do not result in membrane disintegration (V-VIII), as is the case with a lytic crown ether LEC₁₀ (II) or lysozyme (Ill). These results demonstrate that the newly synthesized MAcBCE and MAkBCE compounds do not cause cell lysis or extensive disruption of the cell membrane.

DiSC₃(5) Depolarization Assays

A 2 mL liquid culture of transformed B. subtilis cells was grown at 37° C. in LB medium supplemented with 5 μg/mL kanamycin until OD_{600=0.6} was reached. The cells were collected by centrifugation at 2000 rpm over 3 min. The bacteria were washed twice with an NMDG-MeSO₃ solution (100 mM NMDG, 10 mM HEPES, pH adjusted to 7.4 with methane sulfonic acid). After the last centrifugation step, the bacteria were resuspended until OD₆₀₀=1.0 was reached in the same NMDG-MeSO₃ solution. The working dye solution was a 200 μM solution of DiSC₃(5) (3,3′-dipropylthiadicarbocyanine iodide; Tokyo Chemical Industry) in DMSO. A series of 2 M solutions of NMDG-Cl, NaCl and KCl were prepared containing 10 mM HEPES; the pH was adjusted to pH 7.4 with NMDG, NaOH or KOH, respectively, to avoid any pH changes after their addition. The experiment was initiated with 3 mL of NMDG-MeSO₃ in a quartz cuvette, then 5 μL of the dye solution was added (timepoint t₁, 50 sec) for a 0.3 μM final concentration by the end of experiment. This was followed by the addition of 100 μL of the B. subtilis suspension (timepoint t₂, 100 sec). After 150 sec (timepoint t₃, 250 sec), the fluorescence stabilized to a minimum intensity and 100 μL of the 2 M NMDG-Cl, NaCl or KCl solution was added to reach ˜60 mM final concentration of the salt. The final step was the addition 0.64 μL of a 10 mM stock solution of the desired MAcBCE or MAkBCE compound in TFE (timepoint t₄, 400 sec), for a final concentration of 2 μM. Negative controls were performed by adding the same volume of pure TFE to the cuvette at timepoint t₄. Fluorescence intensity was recorded each second using a Horiba Fluoromax 4 spectrometer (λ_{excitation}=640 nm; λₑₘᵢₛₛᵢₒₙ=670 nm). The solution inside the cuvette was vigorously mixed throughout the experiment by using a magnetic stirrer and the temperature was kept constant at 25° C. For purposes of normalization, the fluorescence intensity was divided by the fluorescence level just before timepoint t₂.

To determine whether MAcBCE or MAkBCE compounds catalyse ion-selective flux, a set of highly bioactive members bearing ten-carbon chain lengths were tested for their ability to depolarize B. subtilis membranes using a test based on the fluorescent dye 3,3-dipropylthiadicarbocyanine (DiSC₃(5)). This set of compounds were selected as four of the most toxic members of the MAcBCE and MAkBCE compound library (5-O-10, 5-A-10, 6-O-10, and 6-A-10; FIG. 3) belong to this group and all are soluble in TFE. The two non-toxic benzo(12-crown-4) scaffold members (4-O-10 and 4-A-10; FIG. 3) were used as controls.

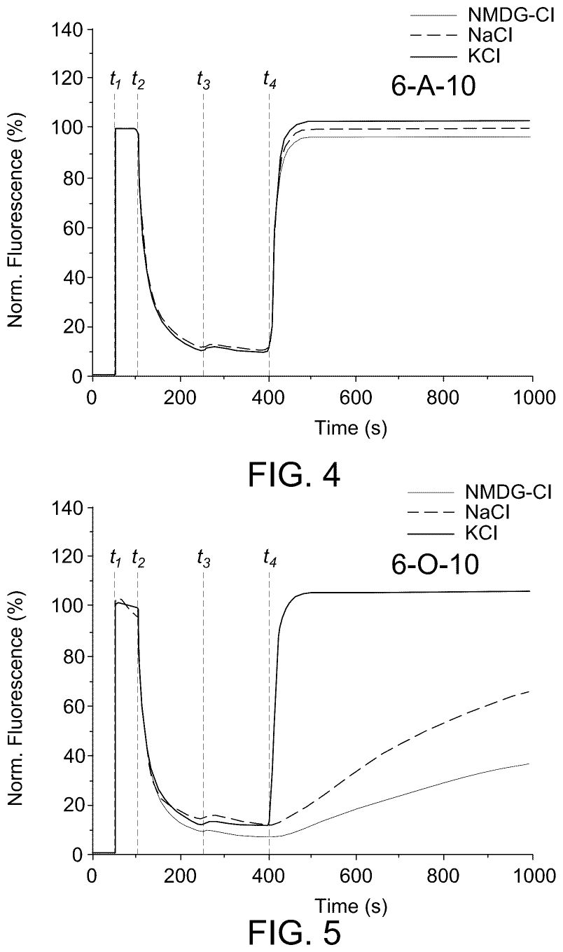

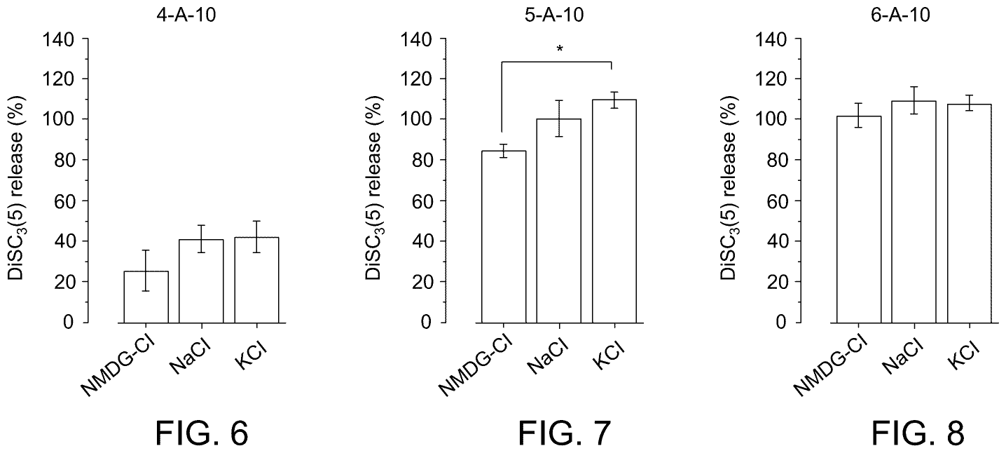

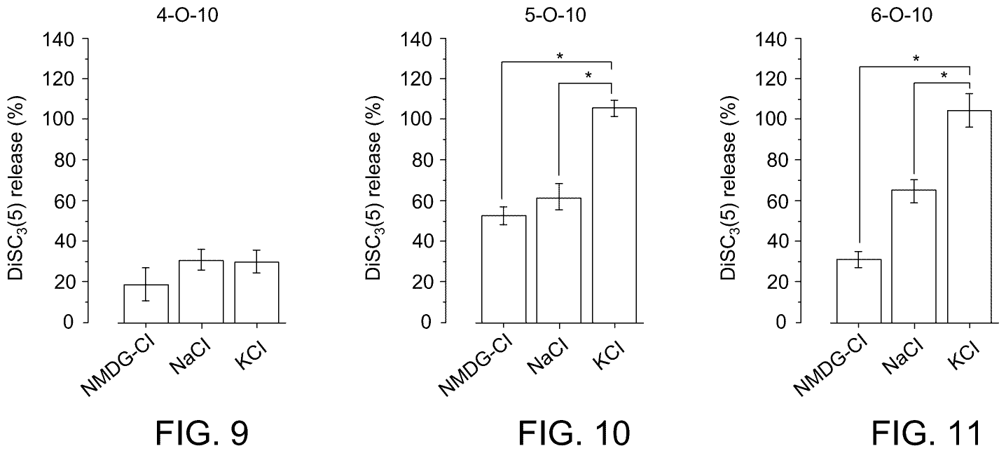

Referring to FIGS. 4-11, MAcBCE and MAkBCE compounds were shown to induce cell membrane depolarization on B. subtilis. FIGS. 4 and 5 show the time course of DiSC₃(5) fluorescence due to the addition of 2 μM 6-A-10 (FIG. 4) and 6-O-10 (FIG. 5). The average of the final fluorescence values (mean±S.E.M.) of at least three experiments are shown in FIGS. 4 and 5 and are similarly plotted for 4-A-10 (FIG. 6), 5-A-10 (FIG. 7), 6-A-10 (FIG. 8), 4-O-10 (FIG. 9), 5-O-10 (FIG. 10), and 6-O-10 (FIG. 11). Experiments were performed in a NMDG-MeSO₃ solution (100 mM NMDG, 10 mM HEPES, pH adjusted to 7.4 with methane sulfonic acid). The events are: t₁, addition of dye; t₂, addition of B. subtilis; t₃, addition of a 2 M NMDG-Cl, 2 M NaCl or a 2 M KCl solution up to a final concentration of 60 mM; t₄, addition of each MAcBCE or MAkBCE compound up to 2 μM. Experiments were repeated at least four times per condition and normalized relative to the fluorescence intensity just before the addition of cells.

The potentiometric dye DiSC₃(5) was used to measure the ability of MAcBCE and MAkBCE compounds to catalyse ion-selective transport. DiSC₃(5) undergoes membrane voltage-dependent partitioning between the intracellular and the extracellular medium. DiSC₃(5) accumulates within a cell with a polarized membrane (negatively charged inside the cell) and is released when the cell membrane is depolarized (positively charged inside the cell). The membrane voltage in B. subtilis is roughly −120 mV, leading to DiSC₃(5) accumulation in the bacteria (timepoint t₂ in FIG. 4A-B). The addition of up to 60 mM KCl, NaCl or N-methyl-d-glucamine chloride (NMDG-Cl) (timepoint t₃ in FIGS. 4 and 5) did not cause substantial changes in the membrane voltage, as revealed by the limited change in the fluorescence. Upon addition of 6-A-10 or 6-O-10 (timepoint t₄ in FIGS. 4 and 5, respectively), a clear increase in fluorescence was observed in all cases, although with differing kinetics. The effects of 6-A-10, 6-O-10 and four other different MAcBCE and MAkBCE compounds on the relative DiSC₃(5) fluorescence after 10 minutes were averaged and plotted in the panels in FIGS. 6-11. In general, the MAcBCE and MAkBCE compounds with the highest toxicities elicited faster DiSC₃(5) efflux, suggesting that they are more efficient at transporting ions (FIGS. 7-8, 10-11), while the changes in the fluorescence after adding the biologically inert 4-O-10 and 4-A-10 were negligible (FIGS. 6 and 9). Interestingly, for compounds 6-O-10, 5-O-10 and 5-A-10, the kinetics of the depolarization is ion dependent; it is fastest in presence of K⁺ followed by Na⁺ and then NMDG⁺. Only in the case of 6-O-10 (FIG. 11) are the differences in kinetics between Na⁺ and NMDG⁺ statistically significant.

Liposomes Preparation

The liposome preparation was based on protocols employed by Jiang et al., Activation of the archaeal ion channel MthK is exquisitely regulated by temperature, Elife 2020, 9, to reconstitute bacterial ion channels in proteoliposomes (giant multilamellar vesicles; GMLV). The method is adapted to produce plain liposomes. A total of 25 mg of asolectin from soybean (Avanti) was dissolved in 1 mL of chloroform, dried under argon, and kept overnight under vacuum. The dried lipids were resuspended using bath sonication in a resuspension solution (250 mM KCl, 30 mM HEPES, 0.1 mM CaCl₂, pH adjusted at 7.6 with KOH) to a final concentration of 15 mg/mL and stored in 30 μL aliquots at −80° C. Aliquots were thawed and placed on a clean glass slide and dried in a desiccator under vacuum at room temperature. The lipids were then rehydrated with 50 μL resuspension solution for more than 2 h to yield the GMLVs.

Electrophysiology

Direct detection of electrophysiological activity in biological membranes is the gold standard for any putative ion channel. The 6-O-10 and 6-A-10 compounds were tested for ion channel activity by using the patch clamp technique in the inside-out configuration on giant multilamellar vesicles (GMLV) of asolectin.

Single-channel recordings were obtained by patch-clamping GMLV. After seal formation, patches were excised to obtain the inside-out configuration, and then the pipette solution and the bath solution were assumed to be “external” and “internal” solutions, respectively. Pipettes of borosilicate capillary glass 2-000-100 (Drummond Scientific Company) were pulled in a horizontal pipette puller P-97 (Sutter Instrument). Pipette resistance was 5-10 MΩ in any of the solutions tested. Data were acquired with an Axopatch 200A (Axon Instruments) amplifier. Current signals were sampled with a 16-bit A/D converter Axon Digi-data 1550B (Axon Instruments) at a sampling rate of 100 kHz and low-pass filtered at 10 kHz. Data was acquired using Clampex 10.7 (Molecular Devices) acquisition software and analysed with Clampfit 10.7 software (Axon Instruments) and Origin. The temperature in the room was approximately 21° C.

For gap-free experiments, symmetrical (same solution in pipette and bath) KCl solution (200 mM KCl, 30 mM HEPES, pH adjusted to 7.4 with KOH), NaCl solution (200 mM NaCl, 30 mM HEPES, pH adjusted to 7.4 with NaOH) or NMDG-Cl solution (200 mM NMDG, 30 mM HEPES, pH adjusted to 7.4 with HCl) were used for both the internal and external solutions. All the traces were recorded at 100 mV for 5 minutes.

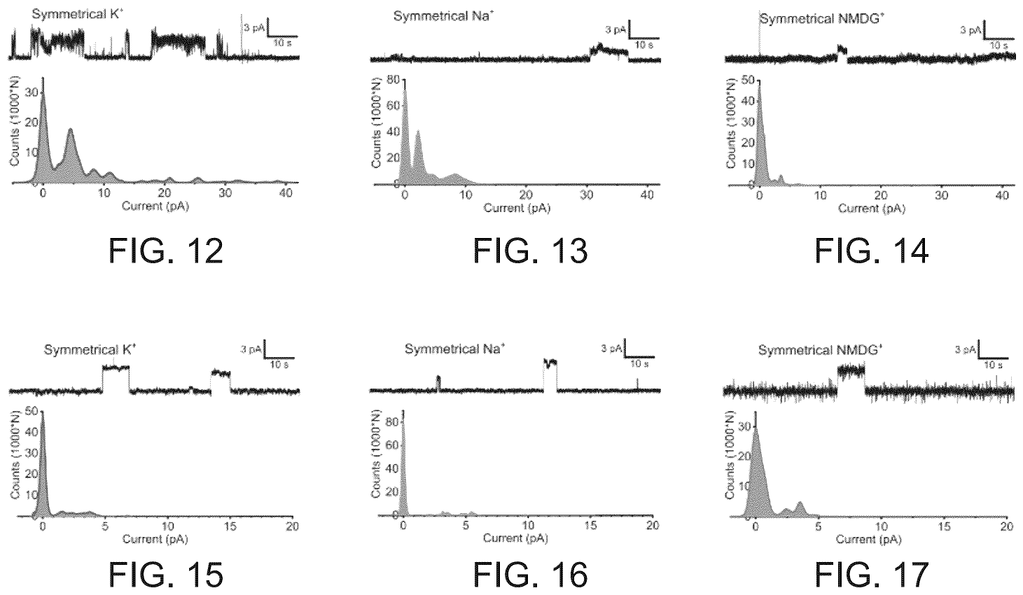

Referring to FIGS. 12-17, the MAcBCE and MAkBCE compounds were shown to elicit ion channel activity in biological membranes. FIGS. 12-14 show single channel activity elicited by 10 μM 6-O-10 in the presence of symmetrical KCl, NaCl and NMDG-Cl solutions, respectively. FIGS. 15-17 show single channel activity elicited by 10 μM 6-A-10 in the presence of symmetrical KCl, NaCl and NMDG-Cl solutions, respectively. Histograms for a total recording time of 900 s are included below every record.

Ion channel activity was measured in the presence of symmetrical 200 mM KCl, NaCl or NMDG-Cl at a concentration of 10 μM for each compound. Three different records of 300 s each were analyzed for every condition shown in (FIGS. 12-17). The application of 0.1% TFE, the maximum concentration used when the MAcBCE and MAkBCE compounds were added, did not elicit any ion channel activity (data not shown). For 6-O-10, it is evident in the raw representative traces, as well as from amplitude histograms, that the single channel activity is highest in the presence of K⁺ and Na⁺ as compared to NMDG⁺. However, the single channel conductance in the presence of Na⁺ and NMDG⁺ is only half that observed in the presence of K⁺. In contrast to 6-O-10 compounds, the single channel activity of 6-A-10 compounds does not vary significantly amongst the ions that were tested here. The observed single channel conductance is also very similar with the different ions.

To estimate the relative permeabilities of various ions, the product of number of channels (N) and open probability (Po) and the conductance from the amplitude histograms must be taken into consideration (Table 1). Unlike measurements from single ion channels, the present analysis is not constrained to recordings from patches that contain single functional channels. Thus, the possibility that the supramolecular assembly of synthetic channels is dynamic, and that new channels form during recordings with different oligomerization states cannot be ruled out. The only parameter that can be controlled is the concentration of the compounds in solution. All measurements reported here were obtained at 10 μM solution concentration. Then, differences in the relative permeability could be due to differences in activity of the compounds in the presence of different ions, different probability of insertion in the lipid bilayer or different open probability once the channel is formed. To quantify the effects of each of these factors is hard to address experimentally.

TABLE 1
Translocation rates of 6-O-10 and 6-A-10
compounds in the presence of various ions

| | | | Trans- |
|---|---|---|---|
| | | | location rate |
| Compound | Solution | NP_{O} | (10⁶/sec) |
| 6-O-10 | KCl | 0.58 ± 0.16 | 43.4 ± 5.8 |
| | NaCl | 0.56 ± 0.10 | 11.3 ± 1.1 |
| | NMDG-Cl | 0.25 ± 0.13 | 7.1 ± 3.4 |
| 6-A-10 | KCl | 0.18 ± 0.02 | 3.2 ± 0.3 |
| | NaCl | 0.16 ± 0.02 | 4.5 ± 0.7 |
| | NMDG-Cl | 0.19 ± 0.12 | 5.0 ± 1.9 |

By integrating the amplitudes histograms and dividing by the total recording time, an estimate of the amount of ions transported per second can be obtained (see Table 1). The 6-O-10 compound in the presence of K⁺ has the highest translocation efficiency. K⁺ permeation via 6-O-10 is 3.5-fold more efficient than Na⁺ and 7-fold more efficient than NMDG⁺. The 6-A-10 compounds are an order of magnitude less efficient in terms of K⁺ transport compared to 6-O-10.

With 6-O-10, particularly in presence of K⁺ ions, multiple unusually high conductance states with large conductance levels are occasionally observed. The conductance levels are so high that these are not compatible with single file transport and suggests that the 6-O-10 may have some lytic activity. They are much less frequent in presence of NMDG⁺ and Na⁺. The 6-A-10 molecules appear to be better-behaved with discrete long-lived conductance states. Overall, the frequency of these high conductance seems to be also directly correlated with their functional activity.

Overall, the single channel data indicates that the ion identity influences the ability of both 6-O-10 and 6-A-10 compounds to form conducting channels. As best understood, ion dependence of supramolecular assembly and the ability to form conducting channels has not been observed previously. Another interesting aspect of the data is the demonstration that these compounds can conduct ions as bulky as NMDG⁺, albeit with low probability. This may suggest an alternative mechanism of ion transport utilizing benzo(crown-ether) compounds.

While the 6-O-10 scaffold preferentially transports K⁺ over Na⁺ or NMDG⁺, it is unclear whether the conducting channels exhibit any ion selectivity in presence of competing ions. This can be determined by measuring the reversal potential under bi-ionic conditions and by applying rapid voltage ramps to measure reversal from open channels. For the determination of permeability ratios, a voltage protocol (detailed below) was applied 100 times to the membrane, while a gradient of ions was set through the membrane (internal solution: 180 mM NaCl, 20 mM KCl, 30 mM HEPES, pH adjusted to 7.4 with NaOH; external solution: 20 mM NaCl, 180 mM KCl, 30 mM HEPES, pH adjusted to 7.4 with NaOH). The protocol started from the holding potential (0 mV), followed by a jump to 100 mV during 100 ms, then a ramp of 1000 ms until −100 mV, maintenance of the voltage at −100 mV for 100 ms before returning to the initial 0 mV holding potential. Calculation of the variance at every isochrone allowed determination of the reversal potential (Vᵣₑᵥ) as the voltage at which the variance is minimized. The permeability ratio for

$${K}^{+} vs {Na}^{+} ( \frac{{p}_{K}}{{p}_{N a}} )$$

was calculated from the Goldman-Hodgkin-Katz (GHK) equation by assuming that reversal potential is the equilibrium membrane voltage reached by a selective membrane in the presence of that gradient of ions. This leads to the equation 1:

$$\frac{{p}_{K}}{{p}_{N a}} = {( {{e}^{\frac{F {V}_{rev}}{R T}} [ {K}^{+} ]}_{i} - {[ {K}^{+} ]}_{e} )}^{- 1} ( {[ {Na}^{+} ]}_{e} - {{e}^{\frac{F {V}_{rev}}{R T}} [ {Na}^{+} ]}_{i} ) \qquad ( 1 )$$

where F is the Faraday constant, R is the gas constant, T is the absolute temperature, [K⁺]ᵢ, and [K⁺]ₑ are the internal and external K⁺ concentrations, respectively, and [Na⁺]ᵢ, and [Na⁺]ₑ are the internal and external Na⁺ concentrations, respectively.

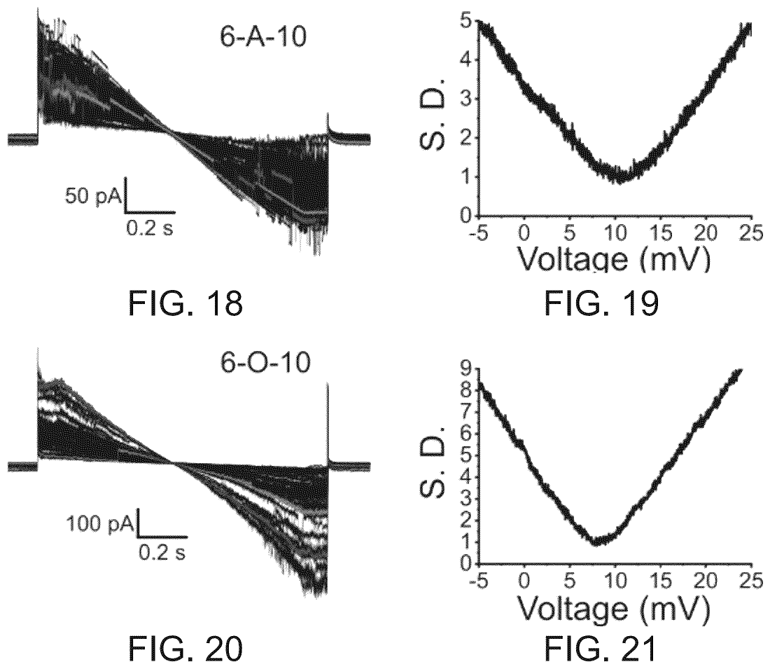

Referring to FIGS. 18-21, it was observed that MAcBCE and MAkBCE scaffold channels do not exhibit any preference for K⁺ over Na⁺. Multiple voltage ramps (N=100) from +100 mV to −100 mV were applied in the presence of 10 μM 6-A-10 (FIG. 18) and 10 μM 6-O-10 (FIG. 20). The reversal potential for each case was determined as that voltage which produces the minimum of variance (FIGS. 19 and 21). In FIGS. 19 and 21, the curves are the variance calculated from experiments in FIGS. 18 and 20, respectively.

In these experiments, the internal concentration of Na⁺ in the pipette was 9-fold higher than the external concentration, whereas the K⁺ gradient was reversed (internal solution: 180 mM NaCl, 20 mM KCl, 30 mM HEPES, pH 7.4; external solution: 20 mM NaCl, 180 mM KCl, 30 mM HEPES, pH 7.4). If the channels are K⁺ selective, the reversal potential will be close to the K⁺ Nernst potential, which is 58 mV. In contrast, if the channels are highly Na⁺ selective, then the reversal potential will be close to the Na⁺ Nernst potential (−58 mV).

The reversal potential can be estimated by plotting current variance with respect to voltage. The estimated reversal potential for 6-A-10 is 8.0±0.8 mV (n=6) (FIG. 19) whereas for 6-O-10 is 6.5±0.6 mV (n=6) (FIG. 21). The estimated reversal potential remains the same when the gradient is reversed (internal solution: 20 mM NaCl, 180 mM KCl, 30 mM HEPES, pH 7.4; external solution: 180 mM NaCl, 20 mM KCl, 30 mM HEPES, pH 7.4). Therefore, it can be concluded that the channels formed by these compounds exhibit no preference for K⁺ or Na⁺ (p_{K}/p_{Na} is 1.4 for 6-A-10 and 1.3 for 6-O-10, respectively).

Although the conducting MABCE channels do not exhibit any preference for Na⁺ vs. K⁺, the single channel recordings show that the open channel probability and the single channel conductance in presence of K⁺ ion is higher than in Na⁺. These differences translate into higher flux in K⁺ compared to Na⁺ and accounts for most of the observed differences between these two ions in the depolarization assay.

What is claimed is:

1. A self-assembling compound for the formation of ion channels in biological membranes, the self-assembling compound being one of a monoacylated benzo(crown-ether) (MAcBCE) compound and a monoalkylated benzo(crown-ether) (MAkBCE) compound.

2. The self-assembling compound of claim 1, wherein the self-assembling compound is a MAcBCE compound having a formula (IA): 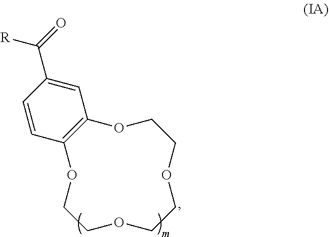
R being a straight chain or branched C₁₋₂₀alkyl, optionally containing unsaturation, that is not substituted with a hydrogen bond donor, and
m being an integer from 1 to 3.

3. The self-assembling compound of claim 2, wherein the MAcBCE compound has a formula (IB): 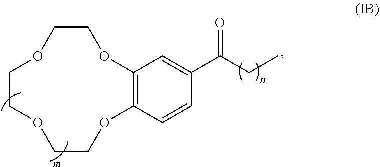
m being an integer from 1 to 3, and
n being an integer from 0 to 19.

4. The self-assembling compound of claim 3, wherein n is an integer from 2 to 9.

5. The self-assembling compound of claim 3, wherein n is an integer selected from the group consisting of 2, 4, 6, 8, and 9.

6. The self-assembling compound of claim 1, wherein the self-assembling compound is a MAkBCE compound having a formula (IIA): 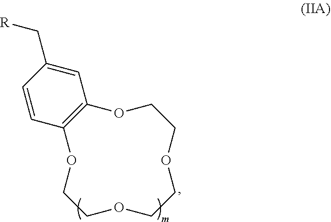
R being a straight chain or branched C₁₋₂₀alkyl, optionally containing unsaturation, that is not substituted with a hydrogen bond donor, and
m being an integer from 1 to 3.

7. The self-assembling compound of claim 6, wherein the MAkBCE compound has a formula (IIB): 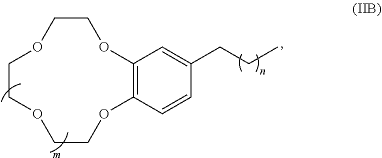
m being an integer from 1 to 3, and
n being an integer from 0 to 19.

8. The self-assembling compound of claim 7, wherein n is an integer from 2 to 9.

9. The self-assembling compound of claim 7, wherein n is an integer selected from the group consisting of 2, 4, 6, 8, and 9.

10. A method of preparing benzo(crown-ether) compounds being monosubstituted with one of an acyl group and an alkyl group, the method comprising:
reacting a carboxylic acid having a formula (III): 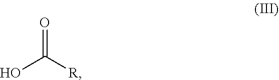
R being a straight chain or branched C₁₋₂₀alkyl, optionally containing unsaturation, that is not substituted with a hydrogen bond donor;
with a benzo(crown-ether) having a formula (IV): 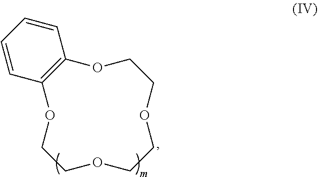
m being an integer from 1 to 3;
in the presence of an acylation acid catalyst to obtain a monoacylated benzo(crown-ether) having a formula (IA): 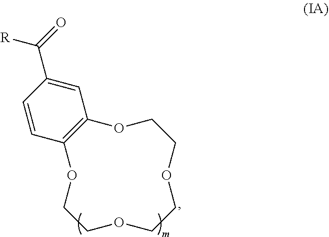
R being a straight chain or branched C₁₋₂₀alkyl, optionally containing unsaturation, that is not substituted with a hydrogen bond donor, and
m being an integer from 1 to 3.

11. The method of claim 10, wherein the acylation acid catalyst comprises Eaton's reagent.

12. The method of claim 11, wherein the reacting the carboxylic acid with the benzo(crown-ether) is performed at a temperature from 10° C. to 100° C.

13. The method of claim 11, wherein the reacting the carboxylic acid with the benzo(crown-ether) is performed at a temperature from 50° C. to 90° C.

14. The method of claim 13, wherein the reacting the carboxylic acid with the benzo(crown-ether) is performed for a duration of less than or equal to 1 hour.

15. The method of claim 10, further comprising reducing the monoacylated benzo(crown-ether) in the presence of a reducing agent to obtain a monoalkylated benzo(crown-ether) having a formula (IIA): 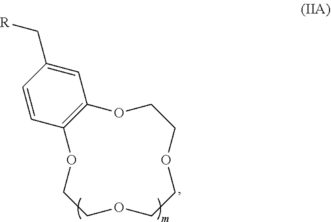
R being a straight chain or branched C₁₋₂₀alkyl, optionally containing unsaturation, that is not substituted with a hydrogen bond donor; and
m being an integer from 1 to 3.

16. The method of claim 15, wherein the reducing agent is a hydrosilane comprising triethyl silane, and the monoacylated benzo(crown-ether) is reduced in a solution with a hydrogenation acid.

17. The method of claim 16, wherein the hydrogenation acid comprises trifluoracetic acid.

18. The method of claim 15, wherein, in each formula (III), (IA), and (IIA), R is a straight chain, saturated C₃₋₁₀alkyl that is not substituted with a hydrogen bond donor.

19. A method of forming an ion channel in a biological membrane, the method comprising combining the membrane with monoacylated benzo(crown-ether) (MAcBCE) compounds, monoalkylated benzo(crown-ether) (MAkBCE) compounds, or a combination thereof, such that the MAcBCE compounds, the MAkBCE compounds, or a combination of the MAcBCE and MAkBCE compounds self-assemble to form the ion channel in the membrane.

20. The method of claim 19, wherein each of the MAcBCE compounds has a formula (IA): 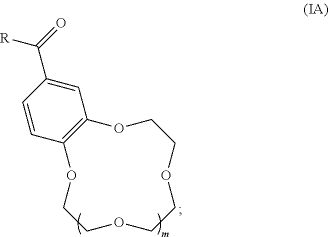
each of the MAkBCE compounds has a formula (IIA): 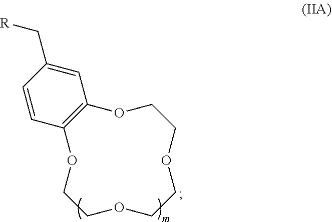
and
in each of the formulas (IA) and (IIA),
R is a straight chain or branched C₁₋₂₀alkyl, optionally containing unsaturation, that is not substituted with a hydrogen bond donor; and
m is an integer from 1 to 3.
